(19) United States
(12) Patent Application Publication
(10) Pub. No.: US 2019/0030002 A1
(43) Pub. Date: Jan. 31, 2019

(54) TREATMENT OF MALIGNANCIES

(71) Applicant: Board of Trustees of Michigan State University, East Lansing, MI (US)

(72) Inventors: Vilma Yuzbasiyan-Gurkan, Ann Arbor, MI (US); Jetze J. Tepe, East Lansing, MI (US)

(21) Appl. No.: 16/049,079

(22) Filed: Jul. 30, 2018

Related U.S. Application Data
(60) Provisional application No. 62/539,049, filed on Jul. 31, 2017.

(51) Int. Cl. A61K 31/4164; A61P 35/00

(52) U.S. Cl. CPC A61K 31/4164; A61P 35/00

(57) ABSTRACT

Compositions and methods are described herein that include imidazole compounds that are useful for treatment of certain cancers, particularly metastatic cancers such as of glioblastomas, osteosarcomas, histiocytic sarcomas, mastocytomas, and sarcomas.

### CROSS-REFERENCE TO RELATED APPLICATIONS

This application claims the benefit from U.S. Provisional Appl. Ser. No. 62/539,049, filed Jul. 31, 2017, which is incorporated by reference as if set forth herein in its entirety.

### BACKGROUND

Canine histiocytic sarcomas (CHS) are aggressive tumors that occur frequently in Bernese mountain dogs and flat-coated retrievers (Kennedy et al., Veterinary Sciences 3(2) (2016)). Clinical signs typically are non-specific and include fever, weight loss, lethargy, and if the tumor presents on a limb, the presence of a noticeably enlarging mass. Canine histiocytic sarcomas can be localized or disseminated. Most commonly canine histiocytic sarcomas are found in the spleen, lymph nodes, lung, bone marrow, skin, brain, and joints of the limbs. Localized canine histiocytic sarcoma involves malignancy in only one organ, whereas disseminated canine histiocytic sarcoma is a multi-system, rapidly progressive disease in which there are tumors present and growing in several different organs. The clinical outcome of these sarcomas is very poor. One study found the median survival time among dogs diagnosed with canine histiocytic sarcoma was about 43 days, regardless of whether the dogs received aggressive chemotherapy and surgery or received no treatment at all (Takahoshi et al., Journal of Veterinary Medical Science 76(5):661-666 (2014)). This is one of the most challenging cancers in dogs, and the prognosis with current therapies is grim. Many other cancers also occur in dogs and cats, and vary in their response to current treatments, but, nearly all succumb to recurrence and develop resistance to current drugs. Therefore, additional approaches and novel drugs are needed for canine and feline cancers.

The ubiquitin-proteasome pathway (UPS) and autophagy pathway are two complementary mechanisms that have evolved to degrade redundant, damaged and misfolded proteins in order to maintain cellular homeostasis (Cohen-Kaplan et al., Int J Biochem Cell Biol 79: 403-418 (2016)). The ubiquitin-proteasome pathway targets the degradation of mostly soluble, short-lived nuclear and cytosolic proteins, whereas the autophagy pathway enables cells to degrade protein complexes, protein aggregates and cellular organelles in a lysosome-dependent mechanism (id.) A well-balanced cross-talk between the two catabolic pathways ensures proper maintenance of cellular function and the cell's amino acid reserve in an energy efficient manner (Park et al., Cell Biochem Biophys 67: 3-13 (2013); Lilienbaum et al., Int J Biochem Mol Biol 4: 1-26 (2013)).

Available therapeutic compounds for cancers such as multiple myeloma (MM) and relapsed/refractory mantle cell lymphoma (MCL) are competitive inhibitors that bind through a covalent, irreversible (or slowly reversible) bond to the N-terminal threonine human proteasome catalytic sites. Examples of small molecules that act as non-competitive proteasome inhibitors are very scarce and exhibit activity only at high micromolar concentrations or non-physiologically relevant concentrations. Unfortunately, more than 97% of multiple myeloma patients develop resistance or become intolerant to the currently available competitive inhibitors within a few years, after which survival is often less than one year.

### SUMMARY

Although some workers suggest that modulation of autophagy is an attractive therapeutic target for treatment of certain diseases, the inventors have tested commercially available compounds and although such testing indicates a possible utility for treatment of canine cancers, such commercially available compounds are highly toxic, require intravenous administration, and/or are prohibitively expensive for use in canines. For at least these reasons, there is a need for new compounds that do not suffer from such drawbacks.

The present disclosure describes compounds that reduce the viability and/or growth of cancerous cells such as glioblastoma, osteosarcoma, histiocytic sarcoma, mastocytoma, and sarcoma cells. The compounds described herein are more active than other commercially available compounds and they can avoid the problems of cellular drug resistance and minimize side effects. Therefore, the compounds provided herein are available at useful (effective) dosages for treatment of cancers.

The present disclosure provides compounds of formula I:

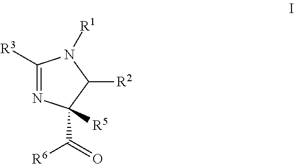

wherein:
R¹ is alkylaryl or arylalkylene, optionally substituted with one to three (1-3) alkyl, alkoxy, halide, or CF₃ groups;
R² is phenyl or -phenyl-N(R⁴)₂, wherein each R⁴ is independently hydrogen, acyl, alkyl, aminoalkyl, formimideamide, thioalkyl, alkylenethiol, aminosulfonyl, sulfonylamine, sulfonylaminoalkyl, aryl, arylalkylene, arylsulfonyl, sulfonylalkylenearyl, cycloalkyl, alkylenecycloalkyl, cycloalkylalkyl, cycloalkylsulfonyl or sulfonylcycloalkyl, wherein the aryl or cycloalkyl is optionally substituted with one to three (1-3) hydroxy, alkoxy or halide groups or
R⁴, and the nitrogen to which it is attached, form a guinidinyl group;
R³ is phenyl or benzyl optionally substituted with halide or alkoxy;
R⁵ is phenyl; and
R⁶ is alkoxy.

In some cases, R² may not be an unsubstituted phenyl; however, in other cases R² may be an unsubstituted phenyl. In some cases, R² is -phenyl-NH—R⁴, but R⁴ is not hydrogen. In some cases, R² is -phenyl-NH—R⁴, but R⁴ is not an alkyl. In some cases, R² is -phenyl-NH—R⁴, but R⁴ is not an aminoalkyl. However, in other cases, R⁴ can be hydrogen, alkyl, or aminoalkyl.

In some cases, R¹ is phenylalkylene. One example of an R¹ group is benzyl.

In some cases, R² is -phenyl-N(R⁴)₂.

In some cases, R³ is phenyl optionally substituted with alkoxy.

In some cases, R⁶ is (C₁-C₃)alkoxy.

One example of a compound of formula I is the TCH-165 compound:

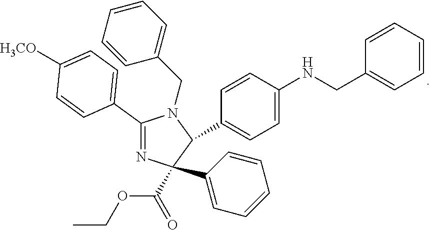 [SMILES: CCOC(=O)[C@]1(c2ccccc2)N=C(c2ccc(OC)cc2)N(Cc2ccccc2)[C@@H]1c1ccc(NCc2ccccc2)cc1]

Another example of a compound of formula I is Imidazole 1 (also referred to as the TCH-013 compound):

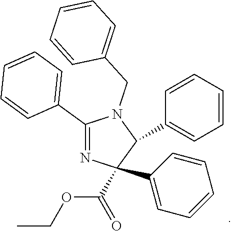 [SMILES: CCOC(=O)[C@]1(c2ccccc2)N=C(c2ccccc2)N(Cc2ccccc2)[C@@H]1c1ccccc1]

This disclosure also describes compositions that include at least one compound of formula I. The compositions can include a carrier, for example, a pharmaceutically acceptable carrier.

This disclosure also describes methods of administering at least one compound of formula I or administering a composition that includes at least one compound of formula I.

For example, the methods can include administering at least one compound of formula I (or a composition thereof) to an animal such as a human, a domesticated animal, or a zoo animal. In some cases the compounds and/or compositions are administered to a domesticated animal. Examples of domesticated animals to whom the compounds and/or compositions can be administered can include dogs, cats, ferrets, horses, donkeys, cattle (e.g., dairy cows), goats, sheep, alpacas, llamas, camels, elephants, birds, and the like.

The animal to whom the compounds and/or compositions can be administered can have a disease or condition. For example, the animals can have cancer. The cancer can be a malignant cancer. Cancers include, without limitation, uterine cancer, urinary bladder cancer, soft tissue sarcoma, prostate cancer, primary peritoneal carcinoma, pancreatic cancer, ovarian cancer, esophageal cancer, malignant mesothelioma, lymphoma, lung cancer, kidney cancer, gastric cancer, gallbladder cancer, colorectal cancer, breast cancer, glioblastoma, glioblastoma astrocytoma, histiocytic sarcoma, lymphoma, osteosarcoma, or a combination thereof. In some cases, the animal to whom the compounds and/or compositions are administered can have a cancer such as a glioblastoma, glioblastoma astrocytoma, osteosarcoma, histiocytic sarcoma, mastocytoma, lymphoma, or sarcoma.

### DESCRIPTION OF THE FIGURES

FIG. 1A is an immunoblot and a bar graph illustrating the amounts of LC3BI and LC3BII (LC3B polypeptides are microtubule-associated protein light chain 3 polypeptides) in U87-MG cells treated with different concentrations of TCH-165 and immunoblotted with LC3B-specific antibody.

FIG. 1B is an immunoblot and a bar graph illustrating the amounts of LC3BI and LC3BII in U87-MG cells over time after treatment with TCH-165 (10 μM) for 0, 6, 12, and 24 h. Cell lysates were immunoblotted with LC3B-specific antibody and anti-GAPDH as a loading control.

FIG. 1C is confocal immunofluorescent images of U87-MG cells, after vehicle-treatment (24 h, left images FIG. 1C1 to 1C3) or TCH-165-treatment (10 μM, 24 h; right images FIG. 1C4 to 1C6) and visualization with LC3B antibodies/Alexa Fluor 488 secondary (green). Blue=Hoechst (fluorescent DNA dye).

FIG. 1D is confocal immunofluorescent images of U87-MG cells, after vehicle-treatment (24 h, left images FIG. 1D1 to 1D3) or TCH-165-treatment (10 μM, 24 h; right images FIG. 1D4 to 1D6) using LAMP1 antibody/Alexa Fluor 594 secondary (red). Blue=Hoechst. All statistical analyses were performed on densitometry data (imageJ) of five individual experiments. Data are graphed as mean±SD and were analyzed by One-Way ANOVA with Bonferroni's multiple comparison test (ns=not significant, *p<0.05, **p<0.01, ***p<0.001).

FIGS. 1A-1D illustrate that compound TCH-165 induces the accumulation of LC3BII and autophagic vacuoles in glioblastoma cells (U87-MG) cells.

FIG. 2A is an immunoblot and a bar graph illustrating conversion of LC3BI to LC3BII as detected with an LC3B-specific antibody. U87-MG cells were treated with either vehicle (DMSO) or TCH-165 (10 μM) for 16 h, followed by treatment with a late stage autophagy inhibitor, bafilomycin A1 (BafAl, V-ATPase inhibitor) (100 nM) for 4 hours.

FIG. 2B is images of U87-MG cells stained with an LC3B-specific antibody detected by confocal immunofluorescence of LC3B in U87-MG cells. U87-MG cells were treated with either vehicle or torin1 (100 nM) for 4 h, followed by TCH-165 (10 μM) for 16 h. LC3B polypeptides were stained with LC3B antibody/Alexa Fluor 488 (green) secondary and the cellular nuclei were stained with Hoechst DNA dye (blue).

FIG. 2C is an immunoblot and a bar graph illustrating amounts of p62 as a function of different concentrations of TCH-165. U87-MG cells were treated with different concentrations of TCH-165 and immunoblotted with p62-specific antibody. GAPDH was immunoblotted as a loading control.

FIG. 2D is an immunoblot and a bar graph illustrating amounts of p62 over time. U87-MG cells were treated with TCH-165 (10 μM) for 0, 6, 12, and 24 h. Cell lysates were immunoblotted with p62-specific antibody and anti-GAPDH as a loading control.

FIG. 2E shows images of U87-MG cells stained with p62 antibodies and visualized by confocal immunofluorescence. U87-MG cells were vehicle-treated (24 h, left) or TCH-165-treated (10 μM, 24 hr; right) and then stained with p62 antibody/Alexa Fluor 488 secondary (red). All statistical analyses were performed on densitometry data (imageJ) of five individual experiments. Data are graphed as mean±SD and were analyzed by One-Way ANOVA with Bonferroni's multiple comparison test or by student t-test for 2 samples (ns=not significant, *p<0.05, **p<0.01, ***p<0.001).

FIGS. 2A-2E illustrate that TCH-165 inhibits autophagic flux in glioblastoma cells (U87-MG) cells.

FIG. 3A is images of U87-MG cells that were transduced with 30 particles per cell of tandem-RFP-GFP-LC3B, cultured for 24 h, and then incubated with either vehicle or TCH-165 (10 μM) for an additional 24 h. Cells were fixed with 4% formaldehyde, counter stained with Hoechts DNA dye, and imaged on a Nikon C2 confocal microscope using standard filter sets for blue dye, GFP and RFP.

FIG. 3B is images U87MG cells treated with either vehicle or TCH-165 (10 μM) for 24 h and immunostained with LC3B specific/Alexa Fluor 488 (green) antibody, LAMP1 specific/Alexa Fluor 594 antibody and Hoechst DNA dye (blue). Images were taken with Nikon A1 confocal microscope (60×).

FIGS. 3A-3B illustrate that TCH-165 inhibits autolysosome formation.

FIG. 4 is an immunoblot showing that TCH-165 does not have any significant effect on the expression of LC3B, p62, beclinl, and ATG5.

FIG. 5A is images of U87-MG cells treated with either vehicle or TCH-165 (10 μM) and images taken with iphone 6S via the oculars of a compound light microscope (10×) at 24 h and 72 h.

FIG. 5B graphically illustrates the viability of glioblastoma cells treated with different concentrations of TCH-165 or TCH-023 for 72 h. MTS reagent was added to quantify cell viability and the absorbance measured at 490 nm 2 h later. Absorbance readings are expressed as percentage of vehicle control for three independent experiments and data are graphed as mean±SD.

FIG. 5C is an immunoblot of glioblastoma cells treated with either vehicle, TCH-165 (10μM) or TCH-023 (10 μM) for 24 h. Cell lysates were immunoblotted to detect LC3B polypeptides and GAPDH.

FIG. 5D is a bar graph showing the viability of glioblastoma cells treated with different concentrations of TCH-165 or temozolomide for 72 h. MTS reagent was then added and absorbance measured at 490 nm 2 h later. Absorbance readings are expressed as percentage of vehicle control for three independent experiments and data are graphed as mean±SD.

FIG. 5E is a plot of % cytotoxicity as a function of log of the concentration of the TCH-165 compound towards U87-MG cells (the LDH assay was used to generate these data).

FIG. 5F is a bar graph illustrating the cytotoxicity of the TCH-165 compound towards U87-MG cells at various concentrations compared to the DNA alkylating agent, temozolomide (TMZ), which is a commercially available chemotherapeutic agent.

### DESCRIPTION

Compounds, compositions, and methods of administration of such compounds and compositions are described herein.

The present disclosure provides compounds of formula I:

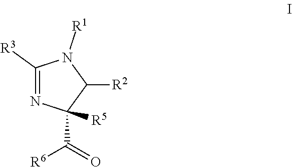

wherein:
R¹ is alkylaryl or arylalkylene, optionally substituted with one to three (1-3) alkyl, alkoxy, halide, or CF₃ groups;
R² is phenyl or -phenyl-N(R⁴)₂, wherein each R⁴ is independently hydrogen, acyl, alkyl, aminoalkyl, formimideamide, thioalkyl, alkylenethiol, aminosulfonyl, sulfonylamine, sulfonylaminoalkyl, aryl, arylalkylene, arylsulfonyl, sulfonylalkylenearyl, cycloalkyl, alkylenecycloalkyl, cycloalkylalkyl, cycloalkylsulfonyl or sulfonylcycloalkyl, wherein the aryl or cycloalkyl is optionally substituted with one to three (1-3) hydroxy, alkoxy or halide groups or
R⁴, and the nitrogen to which it is attached, form a guinidinyl group;
R³ is phenyl or benzyl optionally substituted with halide or alkoxy;
R⁵ is phenyl; and
R⁶ is alkoxy.

In some cases, R² may not be an unsubstituted phenyl; however, in other cases R² may be an unsubstituted phenyl. In some cases, R² is -phenyl-NH—R⁴, but R⁴ is not hydrogen. In some cases, R² is -phenyl-NH—R⁴, but R⁴ is not an alkyl. In some cases, R² is -phenyl-NH—R⁴, but R⁴ is not an aminoalkyl. However, in other cases, R⁴ can be hydrogen, alkyl, or aminoalkyl.

In some cases, R¹ is phenylalkylene. One example of an R¹ group is benzyl.

In some cases, R² is -phenyl-N(R⁴)₂. For example, both R⁴ groups can be a group of the formula -phenyl-NH₂. The group -phenyl-N(R⁴)₂ can also be -phenyl-N(H)benzyl; -phenyl-N(H)C(O)alkyl (e.g., -phenyl-N(H)C(O)CH₃); -phenyl-N(H)C(NH)NH₂; -phenyl-N(H)SO₂phenyl; -phenyl-N(H)SO₂dialkoxyphenyl; -phenyl-N(H)SO₂benzyl; -phenyl-N(H)SO₂cycloalkyl (e.g., cyclopropyl and cyclohexyl); or -phenyl-N(H)SO₂N(alkyl)₂ (e.g., each alkyl can be CH₃).

In some cases, R³ is phenyl optionally substituted with alkoxy.

In some cases, R⁶ is (C₁-C₃)alkoxy.

One example of a compound of formula I is the TCH-165 compound:

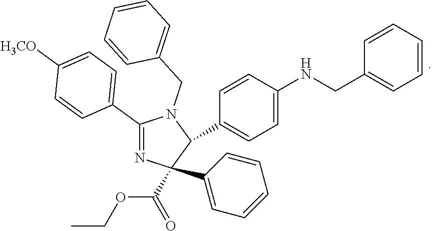 [SMILES: CCOC(=O)[C@]1(c2ccccc2)N=C(c2ccc(OC)cc2)N(Cc2ccccc2)[C@@H]1c1ccc(NCc2ccccc2)cc1]

Another example of a compound of formula I is Imidazole 1 (also referred to as the TCH-013 compound):

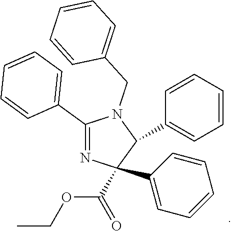 [SMILES: CCOC(=O)[C@]1(c2ccccc2)N=C(c2ccccc2)N(Cc2ccccc2)[C@@H]1c1ccccc1]

Other examples of compounds provided herein include the following:

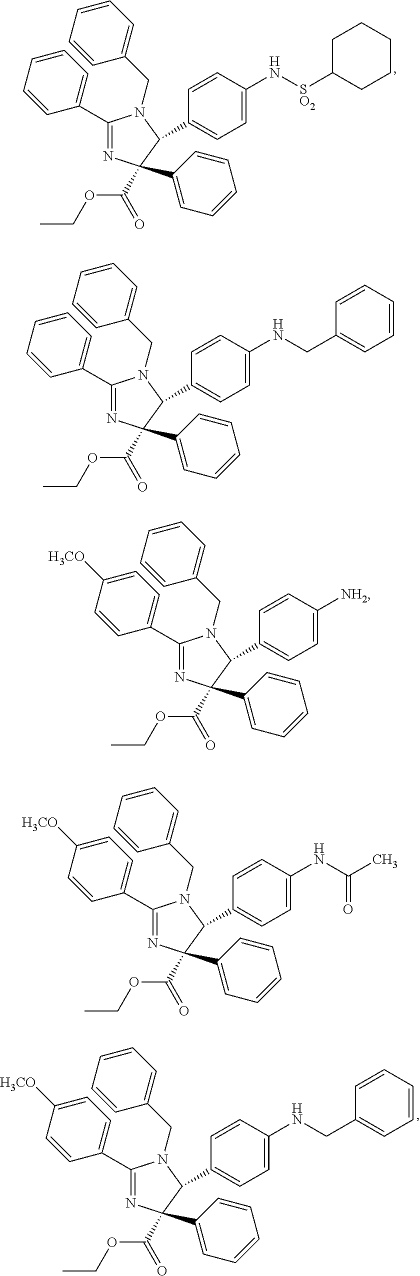

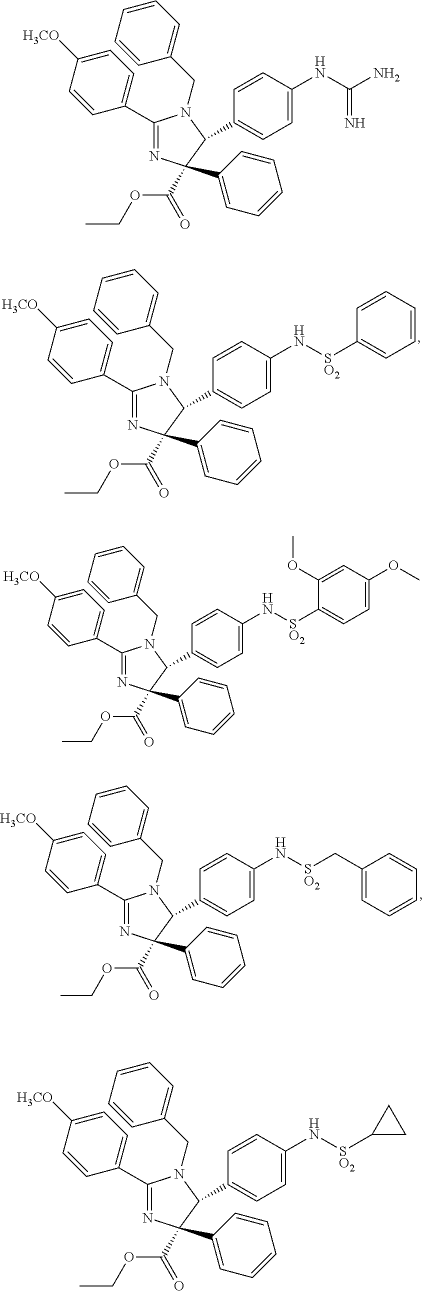 [SMILES: CCOC(=O)[C@]1(c2ccccc2)N=C(c2ccc(OC)cc2)N(Cc2ccccc2)[C@@H]1c1ccc(NC(=N)N)cc1 ; CCOC(=O)[C@]1(c2ccccc2)N=C(c2ccc(OC)cc2)N(Cc2ccccc2)[C@@H]1c1ccc(NS(=O)(=O)c2ccccc2)cc1 ; CCOC(=O)[C@]1(c2ccccc2)N=C(c2ccc(OC)cc2)N(Cc2ccccc2)[C@@H]1c1ccc(NS(=O)(=O)c2ccc(OC)cc2OC)cc1 ; CCOC(=O)[C@]1(c2ccccc2)N=C(c2ccc(OC)cc2)N(Cc2ccccc2)[C@@H]1c1ccc(NS(=O)(=O)Cc2ccccc2)cc1 ; CCOC(=O)[C@]1(c2ccccc2)N=C(c2ccc(OC)cc2)N(Cc2ccccc2)[C@@H]1c1ccc(NS(=O)(=O)C2CC2)cc1]

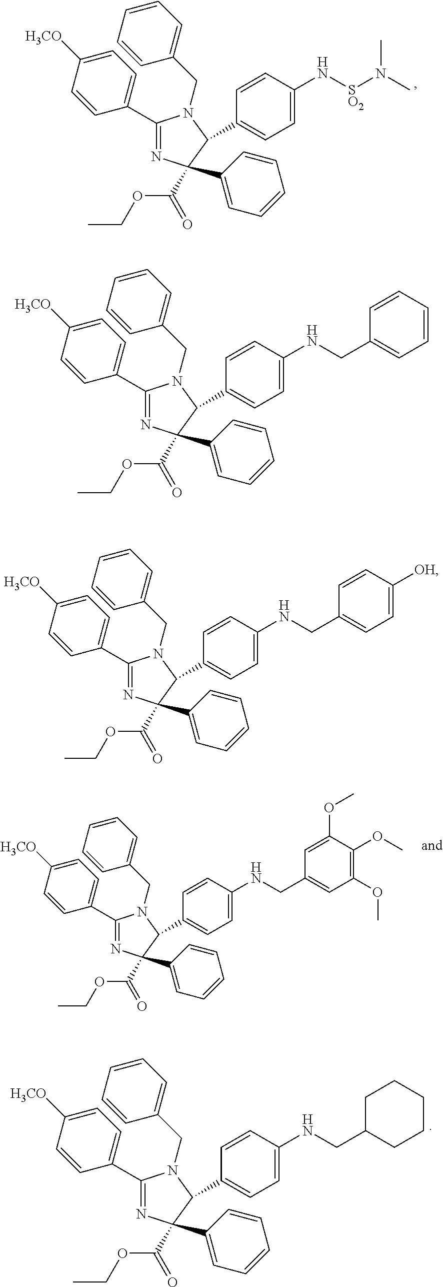 [SMILES: CCOC(=O)[C@]1(c2ccccc2)N=C(c2ccc(OC)cc2)N(Cc2ccccc2)[C@@H]1c1ccc(NS(=O)(=O)N(C)C)cc1 ; CCOC(=O)[C@]1(c2ccccc2)N=C(c2ccc(OC)cc2)N(Cc2ccccc2)[C@@H]1c1ccc(NCc2ccccc2)cc1 ; CCOC(=O)[C@]1(c2ccccc2)N=C(c2ccc(OC)cc2)N(Cc2ccccc2)[C@@H]1c1ccc(NCc2ccc(O)cc2)cc1 ; CCOC(=O)[C@]1(c2ccccc2)N=C(c2ccc(OC)cc2)N(Cc2ccccc2)[C@@H]1c1ccc(NCc2cc(OC)c(OC)c(OC)c2)cc1 ; "and" ; CCOC(=O)[C@]1(c2ccccc2)N=C(c2ccc(OC)cc2)N(Cc2ccccc2)[C@@H]1c1ccc(NCC2CCCCC2)cc1]

### Compositions

The compositions can include any of the compounds described herein, including salt forms, enantiomers and prodrugs of such compounds. The compositions can include a carrier. The compositions can include a pharmaceutically acceptable carrier. By “pharmaceutically acceptable” it is meant that a carrier, diluent, excipient, and/or salt that is compatible with the other ingredients of the formulation and is not deleterious to the recipient thereof. The compositions can be formulated in any convenient form.

The compositions can include the compounds described herein in a “therapeutically effective amount.” Such a therapeutically effective amount is an amount sufficient to obtain the desired physiological effect, such as a reduction of at least one symptom of cancer or other condition.

Symptoms of cancer include fatigue, increased risk of infection, renal failure, anemia, confusion, headaches, lymph node swelling or lumps, loss of appetite, vomiting, diarrhea, and combinations thereof. Symptoms of an inflammatory disease or condition can include joint pain, swollen joints, muscle stiffness, headaches, fever, chills, loss of appetite, systemic pain or aches.

For example, the compounds and methods described herein can inhibit autophagy, decrease the incidence or severity of cancer, and/or decrease cancer cell growth (e.g., decrease cancer cell viability) by 5%, or 10%, or 15%, or 20%, or 25%, or 30%, or 35%, or 40%, or 45%, or 50%, or 55%, or 60%, or 65%, or %70, or 80%, or 90%, 095%, or 97%, or 99%, or any numerical percentage between 5% and 100%.

Administration of the compounds and/or compositions described herein can be in a single dose, in multiple doses, in a continuous or intermittent manner, depending, for example, upon the recipient's physiological condition, whether the purpose of the administration is therapeutic or prophylactic, and other factors known to skilled practitioners. The administration of the compounds and compositions described herein can be essentially continuous over a preselected period of time or can be in a series of spaced doses. Both local and systemic administration is contemplated.

To prepare the composition, the selected compound(s) are synthesized or otherwise obtained, purified as necessary or desired. The compound(s) can be suspended in a pharmaceutically acceptable carrier and/or lyophilized or otherwise stabilized. The compound(s) can be adjusted to an appropriate concentration, and optionally combined with other agents.

The compounds described herein can be administered composition in an amount sufficient to inhibit autophagy. Such an amount can be determined or observed by in vitro or in vivo observation (e.g., quantification) of autophagic vacuole accumulation in the presence and absence of a compound. When the compound increases autophagic vacuole accumulation (relative to a control where the compound is not present), the compound is an inhibitor of autophagy. Dose response curve can be used to evaluate the concentration of compound effective for 50% inhibition of autophagy (IC₅₀).

In another example, autophagy can be evaluated by conversion of microtubule-associated protein 1 light chain 3 (LC3B-I) to LC3B-II via the conjugation of phosphatidylethanolamine (PE) to the C-terminal glycine of LC3B-I. For example, antibodies against LC3B can be used to detect and/or quantify such conversion. The level of LC3B-II correlates with the degree of autophagic vesicle formation, and therefore conversion of LC3B-I to LC3B-II serves as a unique characteristic of autophagosome formation and autophagic activity. A dose response curve can be used to evaluate the concentration of compound effective for 50% conversion of LC3B-I to LC3B-II (IC₅₀).

The absolute weight of a given compound included in a unit dose can vary widely. For example, about 0.001 to about 1 g, or about 0.01 to about 0.5 g, of at least one compound described herein, or a plurality of compounds can be administered. Alternatively, the unit dosage can vary from about 0.002 g to about 1g, from about 0.005 g to about 0.5 g, from about 0.01 g to about 0.25 g, from about 0.02 g to about 0.2 g, from about 0.03 g to about 0.15 g, from about 0.04 g to about 0.12 g, or from about 0.05 g to about 0.1 g.

Daily doses of the compounds can vary as well. Such daily doses can range, for example, from about 0.01 g/day to about 10 g/day, from about 0.02 g/day to about 5 g/day, from about 0.03 g/day to about 4 g/day, from about 0.04 g/day to about 3 g/day, from about 0.05 g/day to about 2 g/day, and from about 0.05 g/day to about 1 g/day.

It will be appreciated that the amount of compound(s) for use in treatment will vary not only with the particular carrier selected but also with the route of administration, the nature of the condition being treated and the age and condition of the patient. Ultimately the attendant health care provider may determine proper dosage.

Thus, one or more suitable unit dosage forms comprising the compounds described herein can be administered by a variety of routes including oral, parenteral (including subcutaneous, intravenous, intramuscular and intraperitoneal), rectal, dermal, transdermal, intrathoracic, intrapulmonary, intraocular, and intranasal (respiratory) routes.

The compounds may also be formulated for sustained release (for example, using microencapsulation, see WO94/07529, and U.S. Pat. No. 4,962,091). A composition may be formulated as a single unit dosage form or into a multitude of dosage forms. The formulations may, where appropriate, be conveniently presented in discrete unit dosage forms and can be prepared by any of the methods available to the pharmaceutical arts. Such methods may include the step of mixing the compound with liquid carriers, solid matrices, semi-solid carriers, finely divided solid carriers or combinations thereof, and then, if desired, introducing or shaping the product into the desired delivery system.

The compositions containing the compounds can be prepared in many forms. Examples include aqueous solutions, suspensions, tablets, hard or soft gelatin capsules, dry powders (e.g. for later reconstitution), liposomes, slow-release formulations, and shaped polymeric gels. Administration can involve oral, parenteral, systemic or local administration of compounds in an aqueous solution or sustained release vehicle.

The compounds can be administered in an oral dosage form. Such an oral dosage form can be formulated as an immediate release, delayed release or sustained release formulation. For example, the compounds can be formulated into a dosage form that releases the compounds into the intestine after passing through the stomach. Such formulations are described in U.S. Pat. No. 6,306,434 and in the references contained therein.

Liquid pharmaceutical compositions may be in the form of, for example, aqueous or oily suspensions, solutions, emulsions, syrups or elixirs, dry powders for constitution with water or other suitable vehicle before use. For example, the compounds can be prepared and stored in dry (e.g., lyophilized) form and then reconstituted into liquid form with an acceptable carrier liquid such as water, saline, buffered saline and the like. Such liquid pharmaceutical compositions may contain conventional additives such as suspending agents, emulsifying agents, non-aqueous vehicles (which may include edible oils), and/or preservatives.

A compound described herein can be formulated for parenteral administration (e.g., by injection, for example, bolus injection or continuous infusion) and can be presented in unit dosage form in ampoules, prefilled syringes, dry form for reconstitution, small volume infusion containers or multi-dose containers.

The compositions can include preservatives, chelating agents, anti-bacterial agents and other therapeutic agents. The compositions may take such forms as suspensions, solutions, or emulsions in oily or aqueous vehicles, and may contain formulatory agents such as suspending, stabilizing and/or dispersing agents. Suitable carriers include saline solution and other materials commonly used in the art.

The compositions can contain proteasome inhibitors such as Bortezomib™ and Carfilzomib™ as well as other therapeutic agents. For example, the compositions can contain other ingredients such as chemotherapeutic agents, anti-inflammatory agents, anti-viral agents, antibacterial agents, antimicrobial agents and/or preservatives.

Examples of additional therapeutic agents that may be used include, but are not limited to: proteasome inhibitors, alkylating agents, such as nitrogen mustards, alkyl sulfonates, nitrosoureas, ethylenimines, and triazenes; antimetabolites, such as folate antagonists, purine analogues, and pyrimidine analogues; antibiotics, such as anthracyclines, bleomycins, mitomycin, dactinomycin, and plicamycin; enzymes, such as L-asparaginase; farnesyl-protein transferase inhibitors; hormonal agents, such as glucocorticoids, estrogens/antiestrogens, androgens/antiandrogens, progestins, and luteinizing hormone-releasing hormone antagonists, octreotide acetate; microtubule-disruptor agents, such as ecteinascidins or their analogs and derivatives; microtubule-stabilizing agents such as paclitaxel (Taxol®), docetaxel (Taxotere®), and epothilones A-F or their analogs or derivatives; plant-derived products, such as vinca alkaloids, epipodophyllotoxins, taxanes; and topoisomerase inhibitors; prenyl-protein transferase inhibitors; and miscellaneous agents such as, hydroxyurea, procarbazine, mitotane, hexamethylmelamine, platinum coordination complexes such as cisplatin and carboplatin; and other agents used as anti-cancer and cytotoxic agents such as biological response modifiers, growth factors; immune modulators, and monoclonal antibodies.

Examples of chemotherapeutic agents that may be co-administered with the compounds described include compounds that induce apoptosis, compounds that reduce the lifespan of cancer cells, compounds that render cells sensitive to stress, as well as any available anti-cancer agents. Examples of agents that can be included in the compositions described herein, or that can be co-administered with the compounds described herein include: aminoglutethimide, amsacrine, anastrozole, asparaginase, bcg, bicalutamide, bleomycin, buserelin, busulfan, campothecin, capecitabine, carboplatin, carmustine, chlorambucil, cisplatin, cladribine, clodronate, colchicine, cyclophosphamide, cyproterone, cytarabine, dacarbazine, dactinomycin, daunorubicin, dienestrol, diethylstilbestrol, docetaxel, doxorubicin, epirubicin, estradiol, estramustine, etoposide, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rituximab, streptozocin, suramin, tamoxifen, temozolomide, teniposide, testosterone, thioguanine, thiotepa, titanocene dichloride, topotecan, trastuzumab, tretinoin, vinblastine, vincristine, vindesine, and vinorelbine.

### Autophagy

Autophagy is an intracellular lysosome-dependent degradation pathway characterized by the formation of a double membrane vacuole, called autophagosome (see, e.g., Kaur et al., Nat Rev Mol Cell Biol 16, 461-472 (2015); Glick et al., J Pathol 221, 3-12 (2010)). In an initial step, an isolated membrane forms through specific autophagy effectors such as the microtubules-associated protein light chain 3 (LC3) that engulfs its targeted protein aggregate or damaged organelle. Elongation and closure of a crescent-shaped structure (phagophore) is mediated by Atg12-Atg5 ubiquitin-like conjugation systems to form an autophagosome. Upon fusion of the autophagosome with the lysosome, several hydrolases, including cathepesins B, D, and L are recruited that act as endopeptidases under the highly acidic condition (pH 4.5-5.0) of the lysosome (see, Mindell Annu Rev Physiol 74, 69-86 (2012)). The low pH is particularly effective for protein degradation and is mainly generated by vacuola-type ATP-ases (V-ATPases), which act as ATP-dependent proton pumps (Reggiori & Ungermann, J Mol Biol 429, 486-496 (2017)).

During autophagy, microtubule-associated protein 1 light chain 3 (LC3B-I) is converted to LC3B-II via the conjugation of phosphatidylethanolamine (PE) to the

C-terminal glycine of LC3B-I. The level of LC3B-II correlates with the degree of autophagic vesicle formation, and therefore conversion of LC3B-I to LC3B-II serves as a unique characteristic of autophagosome formation and autophagic activity (Glick et al., J Pathol 221, 3-12 (2010); Klionsky et al., Autophagy 12, 1-222 (2016)).

Dysregulation of autophagy has been implicated in some pathophysiological disorders including neuro-degenerative disorders and cancer (Jiang et al., Cell Res 24, 69-79 (2014)).

### Treatment

The disclosure describes methods that include administration of any of the compounds described herein, for example, in a composition or dosage form. The compounds described herein include any of the compounds described throughout the application, including salt forms, enantiomers and prodrugs of such compounds. The structural modifications of the compounds described herein can enhance their biological activity. Because the compounds described herein exhibit enhanced activity, only small amounts are needed, and adverse drug reactions are avoided. These compounds are useful for treatment of diseases and conditions such as cancer.

The compounds described herein can be co-administrated with other therapeutic agents.

Examples of cancers that can be treated by administration of the compounds described herein include cancers of the blood, bone, bone marrow, brain, breast, cervix, connective tissues, non-epithelial tissue, intestine, kidney, liver, lung, nervous system, ovaries, pancreas, prostate, skin, testis and combinations thereof. The cancer can be benign or malignant. The cancer can be a hormone-dependent cancer such as a breast, prostate, testicular, or ovarian cancer. The cancer can be a sarcoma, lymphoma, myeloma, or leukemia. In some instances, the cancer is a sarcoma, lymphoma, or glioblastoma.

The cancer treated by the compounds and methods described herein can be a connective tissue cancer, hematological cancer, lymphatic cancer, breast cancer, cervical cancer, ovarian cancer, prostate cancer, testicular cancer, pancreatic cancer, gastrointestinal cancer, neurological cancer, skin cancer, bone cancer, or a combination thereof.

In some cases, the cancer is a glioblastoma, glioblastoma astrocytoma, histiocytic sarcomas, lymphoma, osteosarcoma, or a combination thereof.

Cancers can be treated by administering one or more of the compounds described herein systemically or locally. For example, the compounds can be administered orally, into the blood stream, into a tumor, into a cancerous site, or into the bone marrow. Benign cell growth can also be treated, e.g., warts, by systemic or local administration. In another embodiment, cells can be obtained from a subject, treated ex vivo with the compounds described herein, optionally in combination with other agents or cytotoxins, to remove certain undesirable cells, e.g., cancer cells, and administered back to the same or a different subject.

The compounds and compositions can also be used in conjunction with radiation therapy.

The compounds, compositions, and methods described herein can be used prophylactically or therapeutically. The term “prophylactic” or “therapeutic” treatment refers to administration of one or more compounds (or compositions) to an animal before or after onset of a disease or condition. If it is administered prior to clinical manifestation of the unwanted condition (e.g., disease or other unwanted state of the animal) then the treatment is prophylactic, i.e., it protects the animal against developing the unwanted condition, whereas if administered after manifestation of the unwanted condition, the treatment is therapeutic (i.e., it is intended to diminish, ameliorate or maintain the existing unwanted condition or side effects therefrom). Methods include administering compounds (including enantiomers and salts thereof) in both a prophylactic treatment (e.g. to patients at risk for disease, such as elderly patients who, because of their advancing age, are at risk for cancer, and the like) and therapeutic treatment (e.g. to patients with symptoms of disease or to patients diagnosed with disease).

The compounds described herein exhibit good activity and can be administered at lower dosages with fewer and less severe side effects, adverse drug reactions, hypersensitivities, complications and toxic side effects than currently available imidazoline compounds.

An “adverse drug reaction” refers to a response to a drug that is noxious and unintended and occurs in doses for prophylaxis, diagnosis, or therapy including side effects, toxicity, hypersensitivity, drug interactions, complications, or other idiosyncrasy.

Side effects are adverse symptoms produced by a therapeutic serum level of a compound. For example, the side effect can be produced by a compound's pharmacological effect on an unintended organ system.

A toxic side effect is an adverse symptom or other effect produced by an excessive or prolonged chemical exposure to a compound (e.g., digitalis toxicity, liver toxicity).

Hypersensitivities are immune-mediated adverse reactions (e.g., anaphylaxis, allergy). Drug interactions are adverse effects arising from interactions with other drugs, foods or disease states (e.g., warfarin and erythromycin, cisapride and grapefruit, loperamide and Clostridium difficile colitis).

Complications include conditions caused by a drug (e.g., NSAID-induced gastric ulcer, estrogen-induced thrombosis). The adverse drug reaction may be mediated by known or unknown mechanisms (e.g., Agranulocytosis associated with chloramphenicol or clozapine).

Such side effects, adverse drug reactions, hypersensitivities, complications and toxic side effects can be determined by subject observation, assays or use of animal models available in the art.

### Definitions

The term “substituted” as used herein means that any of the above groups (e.g., alkyl, aryl, arylalkyl, or homocycle) have at least one atom (e.g., a hydrogen) replaced with another substituent. When a double bond is formed to the new substituent (e.g., a new oxo substituent “═O”), two atoms are replaced (e.g., two hydrogen atoms are replaced by an oxo substituent). When substituted, one or more of the groups are “substituents.” For example, the substituent(s) can include one or more hydroxy or alkoxy groups. Other commonly employed groups include halogen(s) such as chloride, bromide or iodide.

Substituents within the context of this disclosure also include, deuterium, tritium, borono, hydroxy, oxo, cyano, nitro, amino, alkylamino, dialkylamino, alkyl, alkoxy, alkylthio, haloalkyl, aryl, arylalkyl, heteroaryl, heteroarylalkyl, heterocycle and heterocyclealkyl, as well as a saccharide, —NRaRb, —NR^{a}C(O)R^{b}, —NR^{a}C(O)NR^{a}NR^{b}, —NR^{a}C(O)OR^{b}, —NR^{a}SO₂R^{b}, —C(O)R^{a}, C(O)OR^{a}, C(O)NR^{a}R^{b}, OC(O)NR^{a}R^{b}, —OR^{a}, —SR^{a}, —SOR^{a}, S(O)₂R^{a}, —OS(O)₂R^{a} and —S(O)₂OR^{a}. R^{a} and R^{b} are the same or different and independently can be hydrogen, halogen, alkyl, cyclolakyl, hydroxyalkyl, haloalkyl, aryl, arylalkyl, alkylaryl, heterocycle, alkylheterocycle or heterocycloalkyl; wherein the aryl, heterocycle or cycloalkyl is optionally substituted with one to three hydroxy, alkoxy or halide groups.

A compound may be described as “unsubstituted” meaning that the compound does not contain extra substituents attached to the compound. An unsubstituted compound refers to the chemical makeup of the compound without extra or replacement substituents, e.g., the compound does not contain protecting group(s).

The term “acyl” as used herein refers to a group containing a carbonyl moiety wherein the group is bonded via the carbonyl carbon atom. The carbonyl carbon atom is also bonded to another carbon atom, which can be part of a substituted or unsubstituted alkyl, alkenyl, alkynyl, aryl, cycloalkyl, heterocyclyl, group or the like. An acetyl (CH₃C(O)—) group is an example of an acyl group.

“Alkyl” means a straight chain or branched, noncyclic or cyclic, unsaturated or saturated aliphatic hydrocarbon containing from 1 to 10 carbon atoms, while the term “lower alkyl” has the same meaning as alkyl but contains from 1 to 6 carbon atoms. The term “higher alkyl” has the same meaning as alkyl but contains from 2 to 10 carbon atoms. Representative saturated straight chain alkyls include methyl, ethyl, n-propyl, n-butyl, n-pentyl, n-hexyl, n-septyl, n-octyl, n-nonyl, and the like; while saturated branched alkyls include isopropyl, sec-butyl, isobutyl, tert-butyl, isopentyl, and the like.

Representative saturated cyclic alkyls, also known as “cycloalkyls,” include cyclopropyl, cyclobutyl, cyclopentyl, cyclohexyl, and the like; while unsaturated cyclic alkyls include cyclopentenyl and cyclohexenyl, and the like. Cyclic alkyls are also referred to herein as “cycloalkyls” or “homocycles” or “homocyclic rings.”

Unsaturated alkyls containing at least one double or triple bond between adjacent carbon atoms (referred to as an “alkenyl” or “alkynyl”, respectively). Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1-butynyl, and the like.

“Alkyloxy,” which is synonymous with “alkoxy,” means an alkyl moiety attached through an oxygen bridge (i.e., —O-alkyl) such as methoxy, ethoxy, and the like.

“Aryl” means an aromatic carbocyclic moiety such as phenyl or naphthyl.

“Aryloxy” means an aryl moiety attached through an oxygen bridge (i.e., —O-aryl). “Arylalkyloxy” means an aryl moiety attached through an alkyloxy bridge (e.g., —O—CH₂-Phenyl).

“Arylamino” means an aryl moiety attached through a nitrogen or amino bridge (e.g., —NH-aryl). “Arylalkylamino” means an aryl moiety attached through an alkylamino bridge (e.g., —NH₂—CH₂-Phenyl). “Arylaminoalkyl” means an aryl moiety attached through an aminoalkyl bridge (e.g., —CH₂—NH₂-Phenyl).

“Aniline” means a phenyl substituted by an amine (-Phenyl-NH₂).

“Heteroaryl” means an aromatic heterocycle ring of 5- to 10 members and having at least one heteroatom selected from nitrogen, oxygen and sulfur, and containing at least 1 carbon atom, including both mono- and bicyclic ring systems. Representative heteroaryls are furyl, benzofuranyl, thiophenyl, benzothiophenyl, pyrrolyl, indolyl, isoindolyl, azaindolyl, pyridyl, quinolinyl, isoquinolinyl, oxazolyl, isooxazolyl, benzoxazolyl, pyrazolyl, imidazolyl, benzimidazolyl, thiazolyl, benzothiazolyl, isothiazolyl, pyridazinyl, pyrimidinyl, pyrazinyl, triazinyl, cinnolinyl, phthalazinyl, and quinazolinyl.

The term “aralkyl,” “arylalkyl,” and “arylalkylene” as used herein refers to alkyl groups as defined herein in which a hydrogen or carbon bond of an alkyl group is replaced with a bond to an aryl group as defined herein. Representative aralkyl groups include benzyl, biphenylmethyl and phenylethyl groups and fused (cycloalkylaryl)alkyl groups such as 4-ethyl-indanyl.

The term “alkylaryl” and “alkaryl” as used herein refers to aryl groups as defined herein in which a hydrogen or carbon bond of an aryl group is replaced with a bond to an alkyl group as defined herein. Representative alkyaryl groups include tolyl, xylyl, and a mesityl group.

The terms “halo,” “halogen,” or “halide” group, as used herein, by themselves or as part of another substituent, mean, unless otherwise stated, a fluorine, chlorine, bromine, or iodine atom.

The term “amine” and “amino” as used herein refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. The term “alkylamino” as used herein refers to an amino group connected to at least one alkyl group, as defined herein, and which can optionally be linked together to form a ring with the nitrogen.

The term “aminoalkyl” refers to alkyl groups as defined herein in which a hydrogen or carbon bond of an alkyl group is replaced with a bond to an amino group as defined herein.

The term “guanidyl” as used herein refers to a group having the formula:

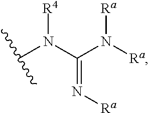

wherein R⁴ is defined herein; and each R^{a} is independently H or alkyl.

The term “alkylthio” and “alkylenethiol” as used herein refers to the group —S-alkyl.

The term “thioalkyl” as used herein refers to an SRa group connected to an alkyl group as defined herein, wherein R^{a} is H or alkyl.

The term “sulfonylamine” or “sulfonylamino” refers to the group —N(R^{a})—SO₂R^{b}, wherein R^{a} and be H or alkyl and R^{b} can be, for example, alkyl, cycloalkyl, aryl or arylalkyl.

The term “aminosulfonyl” refers to the group —SO₂N(R^{a})₂, wherein each R^{a} can be, for example, alkyl, cycloalkyl, aryl or arylalkyl.

The term “sulfonylaminoalkyl” refers to the group -alkyl-N(R^{a})—SO₂R^{b}, wherein R^{a} and be H or alkyl and R^{b} can be, for example, alkyl, cycloalkyl, aryl or arylalkyl.

The term “sulfonylalkylenearyl” and “sulfonylalkylaryl” refers to the group -arylalkyl-SO₂R^{b}, wherein R^{b} can be, for example, alkyl, cycloalkyl or aryl.

The term “alkylenecycloalkyl” and “cycolalkylalkyl” refers to alkyl groups as defined herein in which a hydrogen or carbon bond of an alkyl group is replaced with a bond to a cycloalkyl group as defined herein.

The term “cycloalkylsulfonyl” refers to the group —SO₂R^{b}, wherein Rb is cycloalkyl.

The term “arylsulfonyl” refers to the group —SO₂R^{b}, wherein R^{b} is aryl.

“Homocycle” (also referred to herein as “homocyclic ring”) means a saturated or unsaturated (but not aromatic) carbocyclic ring containing from 3-7 carbon atoms, such as cyclopropane, cyclobutane, cyclopentane, cyclohexane, cycloheptane, cyclohexene, and the like.

“Isomers” means any of two or more substances that are composed of the same elements in the same proportions but differ in the three-dimensional arrangement of atoms; this term includes enantiomeric (i.e., mirror images) and diastereomeric isomers.

“Subject” means any animal, for example, a human patient, livestock, zoo animal, or domestic pet.

As used herein, the terms “treat” and “treating” are not limited to the case where the subject (e.g. patient) is cured and the disease is eradicated. Rather, the disclosure also contemplates treatment that merely reduces symptoms, and/or delays disease progression. For example, treatment can reduce the symptoms of a disease or condition (e.g., cancer) by 5%, or 10%, or 15%, or 20%, or 25%, or 30%, or 35%, or 40%, or 45%, or 50%, or 55%, or 60%, or 65%, or %70, or 80%, or 90%, 095%, or 97%, or 99%, or any numerical percentage between 5% and 100%.

As used herein, the terms “prevent” and “preventing” include the inhibition of the recurrence, spread or onset. It is not intended that the disclosure be limited to complete prevention. Instead, the onset can be delayed, or the severity of the disease can be reduced.

The term “manage” when used in connection with a disease or condition means to provide beneficial effects to a subject being administered with a prophylactic or therapeutic agent, which does not result in a cure of the disease. In certain embodiments, a subject is administered with one or more prophylactic or therapeutic agents to manage a disease so as to prevent the progression or worsening of the disease.

“Cancer” means any of various cellular diseases with malignant neoplasms characterized by the proliferation of anaplastic cells. It is not intended that the diseased cells must actually invade surrounding tissue and metastasize to new body sites. Cancer can involve any tissue of the body and have many different forms in each body area. Many cancers are named for the type of cell or organ in which they start.

Within the context of certain embodiments, whether “cancer is reduced” may be identified by a variety of diagnostic manners known to one skill in the art including, but not limited to, observation the reduction in tumor size, or number of cancer cells, or number of tumor masses, or an increase of apoptosis of cancer cells. Cancer can be reduced (cell death or apoptosis increased) by more than a 5%, or more than 10%, or more than 20%, or more than 25%, or more than 50%. Such a reduction in cancer or increase in apoptosis of cancer cells can be observed after administration or exposure to selected compound (e.g., an imidazoline compound described herein) compared to a control subject or sample not administered or contacted without the compound. Reduction in cancer or increase in apoptosis of cancer cells can also be identified by a change in relevant biomarker or gene expression profile, such as PSA for prostate cancer, her2 for breast cancer, or others. For example, reduction of cancer may be identified in vitro using the following conditions for evaluation of apoptosis: i) cells (e.g., sarcoma cells, glioblastoma cells, Jurkat human T-cell leukemia) are passed into flasks (250 mL, 75 cm2) with 20 mL of supporting media; ii) after incubation at 37° C. with 5% CO₂, sample compound (or for a control, no compound) is added to a flask at a selected concentration (e.g., 1 nanomolar to 1 millimolar), and cells are incubated for another day; iii) cells are treated with 10 μM camptothecin and incubated with SYTOX Green reagent and annexin V allophycocyanin (APC) conjugate (Invitrogen) and iv) Flow cytometry at 488 nm and 633 nm excitation. In cells undergoing apoptosis, phosphatidylserine (PS) is transferred from the cytoplasmic surface of the cell membrane to the outer leaflet. Annexin V has a high affinity for phosphatidylserine and dye conjugates provide an indication of apoptosis by phosphatidylserine exposure and membrane integrity.

As used herein, the term “salts” and “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic groups such as amines; and alkali or organic salts of acidic groups such as carboxylic acids. Pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic, and the like.

Pharmaceutically acceptable salts can be synthesized from the parent compound which contains a basic or acidic moiety by conventional chemical methods. In some instances, such salts can be prepared by reacting the free acid or base forms of these compounds with a stoichiometric (or larger) amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, the disclosure of which is hereby incorporated by reference.

The term “prodrug” means a derivative of a compound that can hydrolyze, oxidize, or otherwise react under biological conditions (in vitro or in vivo) to provide an active compound, particularly a compound of the instant disclosure. Examples of prodrugs include, but are not limited to, derivatives and metabolites of a compound of the instant disclosure that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Specific prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by Burger's Medicinal Chemistry and Drug Discovery 6th ed. (Donald J. Abraham ed., 2001, Wiley) and Design and Application of Prodrugs (H. Bundgaard ed., 1985, Harwood Academic Publishers GmbH).

Values expressed in a range format should be interpreted in a flexible manner to include not only the numerical values explicitly recited as the limits of the range, but also to include all the individual numerical values or sub-ranges encompassed within that range as if each numerical value and sub-range were explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

The term “about” as used herein can allow for a degree of variability in a value or range, for example, within 10%, within 5%, or within 1% of a stated value or of a stated limit of a range.

This application is related to U.S. Pat. No. 6,878,735, U.S. Pat. No. 7,345,078, U.S. Pat. No. 7,528,161, U.S. Pat. No. 7,652,056, U.S. Pat. No. 7,858,808 and U.S. Pat. No. 8,252,942, the contents of each of which are specifically incorporated by reference herein in their entireties.

The terms and expressions that have been employed are used as terms of description and not of limitation, and there is no intention in the use of such terms and expressions of excluding any equivalents of the features shown and described or portions thereof, but it is recognized that various modifications are possible within the scope of the embodiments of the present disclosure. Thus, it should be understood that although the present disclosure has been specifically disclosed by specific embodiments and optional features, modification and variation of the concepts herein disclosed can be resorted to by those of ordinary skill in the art, and that such modifications and variations are considered to be within the scope of embodiments of the present disclosure

The following working examples are provided for the purpose of illustration only and specifically point out certain embodiments, and are not to be construed as limiting in any way the remainder of the disclosure. Therefore, the examples should be construed to encompass any and all variations which become evident as a result of the teaching provided herein.

### EXAMPLES

The specification can be better understood by reference to the following non-limiting examples, which are offered by way of illustration. Compound synthesis

The synthesis of the imidazolines can be accomplished via a dipolar cycloaddition reaction of an oxazol-5(4H)-one and a subsequent esterification of the carboxylic acid (Scheme 1). See, e.g., Kahlon et al., Bioorg. Med. Chem. 17: 3093-3103 (2009); Peddibhotla et al., Org. Lett. 4: 3533-3535 (2002); Peddibhotla et al., Synthesis-Stuttgart 1433-1440 (2003); Sharma & Tepe, Org. Lett. 7: 5091-5094 (2005).

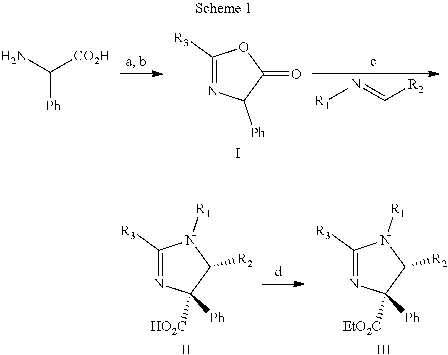

For synthesis of imidazolines via a dipolar cycloaddition reaction as illustrated in Scheme 1, reagents and conditions can be employed such as (a) Na₂CO₃, H₂O/1,4-dioxane, R₃COCl, where the reaction proceeds at room temperature; (b) trifluoroacetic anhydride (TFAA), dichloromethane (DCM), at room temperature; (c) imine (R¹-N═CH—R²), trimethylsilyl chloride (TMSCI), dichloromethane, with reflux; and (d) ethyl-3-(3-dimethylaminopropyl)carbodiimide-HCl (EDCI.HCl), dimethylaminopyridine (DMAP), ethanol (EtOH), and dichloromethane at room temperature.

Acylation of phenylglycine with the selected R³-containing acid chloride followed by cyclic dehydration using trifluoroacetic anhydride can provide the oxazolone (I). See, e.g., Fisk et al., Chem. Soc. Rev. 36: 1432-1440 (2007). The oxazolone can undergo a munchnone-type cyclo-addition reaction in the presence of Lewis acids, such as trimethylsilyl chloride (TMSCI), with and imine such as R¹—N═CH—R² to yield the functionalized imidazoline scaffold (II) as a single diastereomer. See, Sharma & Tepe, Org. Lett. 7: 5091-5094 (2005); Peddibhotla & Tepe, Synthesis 9: 1433-1440 (2003); Peddibhotla et al., Org. Lett. 4: 3533-3535 (2002). The cycloaddition reaction places a carboxylic acid in the C-4 position of the imidazoline scaffold (II).

However, the free carboxylic acid on the imidazoline scaffold (II) is metabolically unstable so functionalization of this group was examined. The formation of an ester (III) can be accomplished using a carbodiimide (1-ethyl-3-(3-dimethylaminopropyl carbodiimide, EDCI) and dimethylaminopyridine (DMAP) to yield esters without the decarboxylation and aromatization problems. The imidazolines were able to tolerate multiple different ester groups without gaining or losing activity.

### Materials and Reagents

Chloroquine, torin-1, mouse monoclonal anti LAMP1, rabbit monoclonal antibodies against LC3B, P62/SQTM1, Beclin-1, ATG-5, and ATG-12, anti-rabbit IgG (H+L), F(ab′)2 Fragment (Alexa Fluor 488 Conjugate), anti-mouse IgG (H+L), F(ab′)2 Fragment (Alexa Fluor 594 conjugate), were obtained from Cell Signaling Technology (Danvers, Mass.). Rabbit polyclonal GAPDH-HRP and goat anti-rabbit-HRP were purchased from Santa Cruz Biotechnologies (Santa Cruz, Calif.). LysoSensor Yellow/Blue dextran, 10,000 MW, Anionic, Fixable; CellLight Lysosomes-RFP, BacMam 2.0 (LAMP1-RFP) and Premo Autophagy Tandem Sensor RFP-GFP-LC3B were purchased from Thermofisher Scientific Incorporation. CelLytic M buffer, sigmafast inhibitor cocktail, bafilomycin A1 and fetal bovine serum were obtained from Sigma Aldrich (St. Louis, Mo.). AQueous One Solution Cell Proliferation Reagent (MTS) and CytoTox 96® Non-Radioactive Cytotoxicity assay (LDH) were obtained from Promega. Dulbecco's Modified Eagle's Medium (DMEM), 0.25% Trypsin-EDTA, penicillin/streptomycin, and buffers were from Life Technologies. Nitrocellulose membrane, Clarity western ECL reagent, blocking grade milk, and precast SDS gels were bought from Bio Rad (Hercules, Calif.). Embryonic kidney cells (HEK293T) was a gift from Dr. Benita Sjogren, Department of Pharmacology & Toxicology, Michigan State University, while glioblastoma astrocytoma (U87-MG) cell lines were obtained from ATCC.

### Cell Culture

Human embryonic kidney cells (HEK293T) were maintained in Dulbecco's Modified Eagle's Medium (DMEM) supplemented with 10% Fetal Bovine Serum, and 100 U/mL Penicillin/Streptomycin. Glioblastoma cells (U87-MG) were maintained in DMEM supplemented with 10% Fetal Bovine Serum, and 100 U/mL Penicillin/Streptomycin.

### LC3B/P62/ATG5/ATG12/beclin1/GAPDH Immunoblot

U87-MG cells at 70% confluency were treated with test compounds at the reported concentration and time. Cells were washed with warm PBS and trypsinized with 0.25%Trypsin-EDTA. Samples were collected in chilled PBS, pelleted and washed twice with chilled PBS. Cells were then lysed with celLytic buffer supplemented with 1% sigma fast inhibitor cocktail. Total protein was quantified by BCA reagent, and 20 μg resolved on a 12% (LC3B) or 30 μg on a 4-12% (p62, ATGS, beclin1) Tris-glycine SDS-PAGE, and immunoblotted with antigen specific antibody (1:1000), and goat anti-rabbit HRP (1:1000). Blots were developed with ECL western reagent and imaged with x-ray film.

### Confocal Immunofluorescent

U87-MG cells were plated in complete medium at 2.5×10⁵ cells/well of a four-chamber glass slide overnight. Fresh medium (500 μL) containing the test compound was added and allowed to incubate for the desire time under cell culture conditions. Cells were washed with chilled immunofluorescent (IF) wash buffer (1× PBS, pH 8.0) and fixed with methanol at −20° C. for 10 minutes. Cells were then washed 2× with IF wash buffer and incubated in blocking buffer (1× PBS, 5% normal goat serum, 0.3% Triton X-100 pH 8.0) for 1 h at RT. Blocking buffer was replaced with primary antibody diluted in dilution buffer(1× PBS, 1% BSA, 0.3% Triton X-100 pH 8.0) overnight (1:200) at 40° C. Primary antibody was washed off 3× and incubated in anti-rabbit Alexa Fluor 488 (1:500) and/or anti-mouse Alexa Fluor 594 for lh in the dark, at RT. Secondary antibodies were then washed off (3×) and nuclei stained with Hoechst (4 μg/mL) at RT, for 20 minutes. DNA dye was rinsed off and slides mounted in Prolong Gold and allowed to cure overnight at RT, in the dark. Slides were sealed with nail polish, and LC3B, LAMP1 and p62 punctae detected with a 60× Plan Apo oil objective on a Nikon C2+ or Nikon A1 confocal laser microscope.

### Confocal Live Imaging

U87-MG or HEK293T cells cultured on cover glass slide were transduced with 30 particles per cell of tandem-RFP-GFP-LC3B and cultured for 24 h. Cells were then incubated with either vehicle, TCH-165 (10 μM), chloroquine (100 μM) or leupeptin A (200 μM) for a further 24 h. Cells were either imaged directly or fixed with 4% formaldehyde and counter stained with Hoechst DNA dye. Images were taken on an upright Nikon Al confocal microscope using a 60× Plan Apo oil objective with standard filter sets for GFP, RFP and DAPI.

### Lysosome De-Acidification Live Imaging

HEK293T cells cultured on cover glass slides were transduced with 30 particles per cell of LAMP1-RFP and cultured for 24 h. Cells were then incubated with either vehicle, TCH-165 (10 μM) or chloroquine (100 μM) for 12 h. LysoSensor Yellow/Blue dextran, 10,000 MW, Anionic (1 mg/mL) was then added for a further 12 h and cells imaged with an upright 60× Plan Apo oil objective on a Nikon A1 confocal microscope using standard filter sets for RFP, blue and yellow dyes.

### Cell Viability Assay (MTS Assay)

U87-MG cells were seeded in a clear 96-well plate at a density of 1.0×104 cells per well. Cells were treated with different concentrations of TCH-165 or temozolomide for 72 h. MTS solution (20 μL) was then added and incubated under cell culture conditions for 2 h. Absorbance was read at 490 nm and expressed as a percentage of vehicle control.

### Cytotoxicity Assay (LDH Assay)

U87-MG cells were seeded in a clear 96-well plate at a density of 1.0×104 cells per well. Cells were treated with different concentrations of TCH-165 or temozolomide for 72 h. Culture medium was then assayed for lactate dehydrogenase (LDH) activity according to the manufacturer's protocol. Briefly, cells for maximum toxicity were treated with 10 μL of lysis buffer 45 minutes prior to enzyme assay. Culture media (50 μL) were then transferred to new 96 well plate and 50 μL of substrate mix added. The reaction mixtures were incubated at RT protected from light for 30 minutes. Stop solution (50 μL) was then added and absorbance read at 490 nm. Toxicity was expressed as a fraction of sample treated with lysis buffer.

### Statistical Analyses

Data are presented as mean±standard deviation of at least three independent experiments. Western blots were quantified with imageJ and statistical analysis done with GraphPad Prism 5 software. Unpaired Student's t-test was used for two samples while one-way analysis of variance with post hoc Bonferroni test was used for multiple comparisons of means.

### Example 1

### TCH-165 Increases the Number of Autophagic Vacuoles

This Example describes experiments showing that certain imidazoline compounds can modulate autophagic flux.

One trans-imidazoline tested was the TCH-165 compound, which is structurally related to an inactive control TCH-023 compound. The structures of these compounds are shown below.

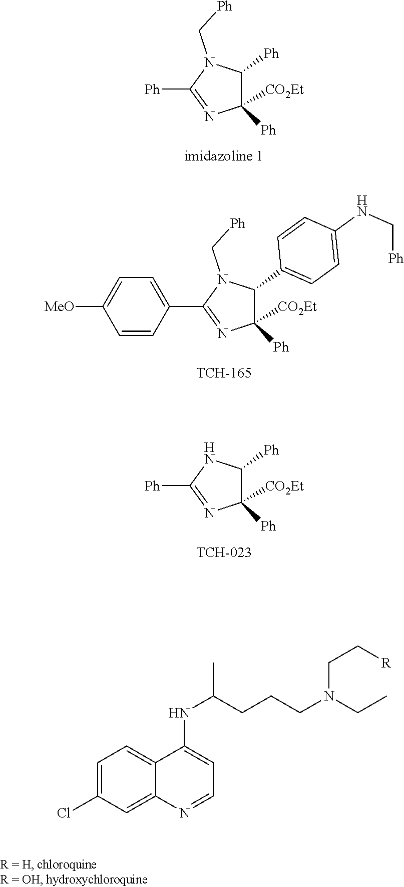

For this study, LC3B, p62, beclin-1, Atg12 and 5 were evaluated as biomarkers for changes of autophagic flux upon exposure to selected imidazolines.

During autophagy, microtubule-associated protein 1 light chain 3 (LC3B-I) is converted to LC3B-II via the conjugation of phosphatidylethanolamine (PE) to the C-terminal glycine of LC3B-I. The level of LC3B-II correlates with the degree of autophagic vesicle formation, and therefore conversion of LC3B-I to LC3B-II serves as a unique characteristic of autophagosome formation and autophagic activity.

To determine whether TCH-165 affects LC3B-II formation, glioblastoma astrocytoma (U87-MG) cells were treated with TCH-165 and the level of LC3B-II monitored by immunoblot. U87-MG cells were incubated with different concentrations of TCH-165 and immunoblotted with LC3B-specific antibody. U87-MG cells were also treated with a specific amount of TCH-165 (10 μM) and evaluated at 0, 6, 12, and 24 hours. Cell lysates were immunoblotted with LC3B-specific antibody and anti-GAPDH as a loading control.

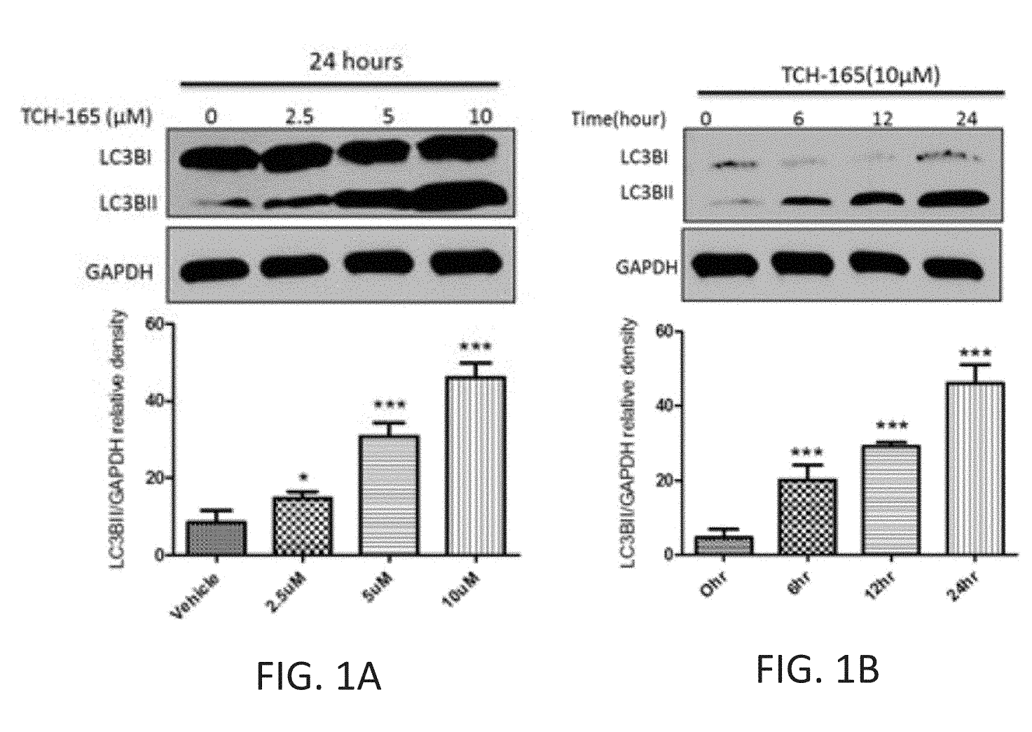

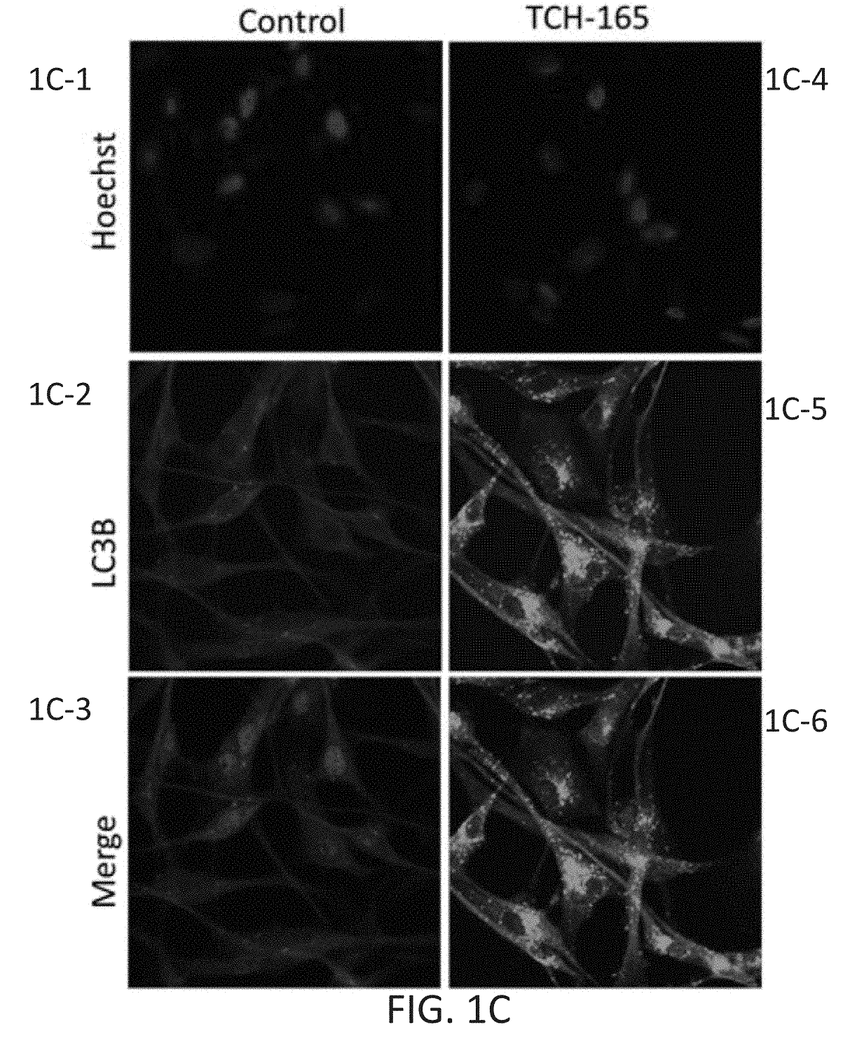

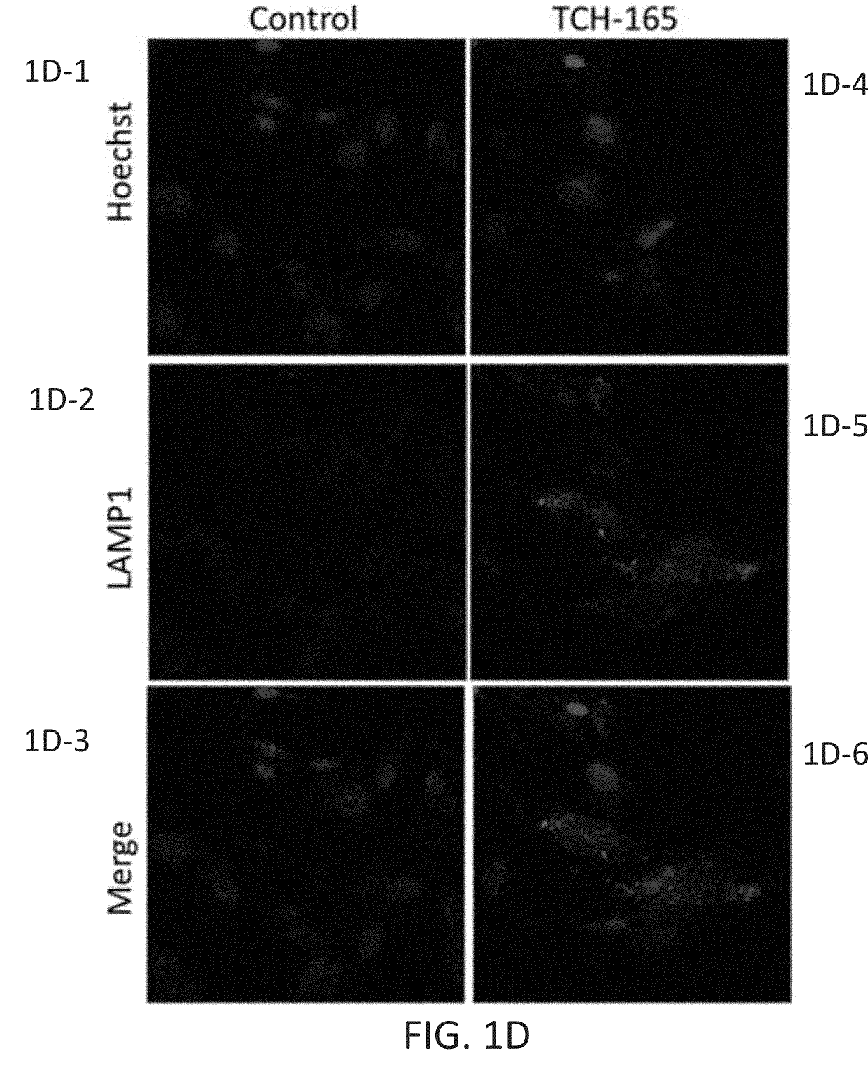

As shown in FIG. 1, TCH-165 increases LC3B-II levels in a concentration (FIG. 1A) and time (FIG. 1B) dependent manner. These data indicate that TCH-165 increases autophagic vesicle formation.

U87-MG cells were evaluated by confocal immunofluorescent analysis after TCH-165-treatment (10 μM, 24 h) using LC3B antibody/Alexa Fluor 488 secondary (green), and Hoechst (blue fluorescent DNA dye) staining.

LC3B-I displayed a diffuse staining pattern within the cytoplasm, while LC3B-II appeared as small punctae in autophagic vacuoles (AVs). To confirm that TCH-165 modulates autophagy, immunofluorescence was used to verify the formation of LC3B positive vacuoles. U87-MG cells were treated with either vehicle (DMSO) or TCH-165 (10 μM) for 16 hours and stained with LC3B specific antibody and Alexa Fluor 488. Confocal laser scanning microscopy (CLSM) revealed a significant increase in the number of LC3B punctate in TCH-165 treatment cells (FIG. 10).

U87-MG cells were evaluated by confocal immunofluorescent analysis after TCH-165-treatment (10 μM, 24 h) using an antibody against lysosome associated membrane protein (LAMP)-1 labeled with Alexa Fluor 594 secondary (red) and Hoechst (blue fluorescent DNA dye) staining.

Staining the cells with antibody against lysosome associated membrane protein (LAMP)-133 showed that the cells developed increased numbers of lysosomes after TCH-165 treatment (FIG. 1D). The structurally related imidazoline, TCH-023, had no effect on LC3B-II accumulation, indicating that the effects on autophagic flux are structure-specific and not a general feature of this class of compounds. Taken together, these data show that TCH-165 increases the number of autophagic vacuoles.

### Example 2

### TCH-165 is a Late Stage Autophagy Inhibitor

Increased levels of LC3B-II and accumulation of autophagic vacuoles could result from increased upstream autophagosome formation (activation) or impaired downstream degradation of basal level autophagosome (inhibition). To distinguish these two possibilities, LC3B-II accumulation was evaluated in the presence of the late stage autophagy inhibitor, bafilomycin A1 (BafA1, V-ATPase inhibitor).

U87-MG cells were treated with either vehicle (DMSO) or TCH-165 (10 μM) for 16 h, followed by treatment with bafilomycin A1 (100 nM) for 4 h. Conversion of LC3BI to LC3BII was detected by immunoblot with LC3B-specific antibody.

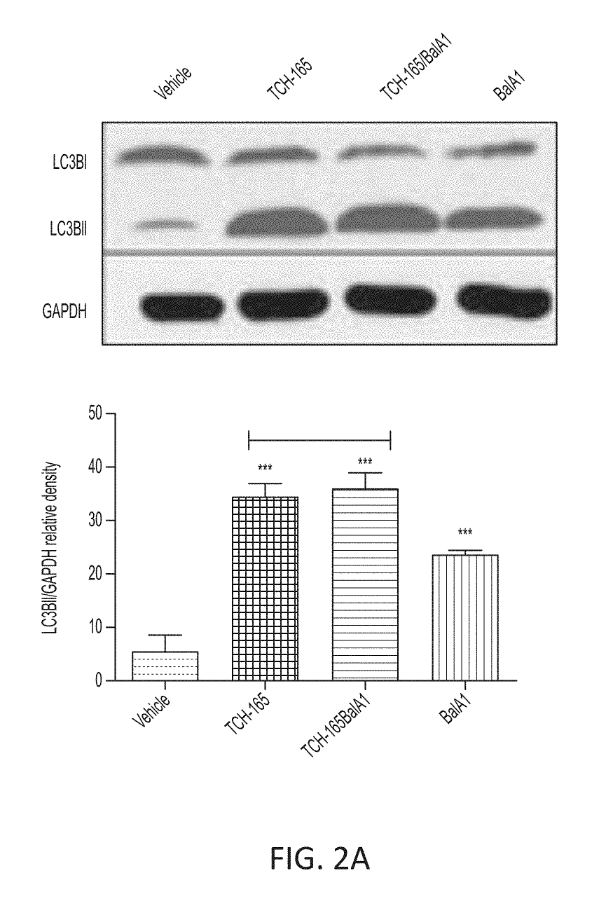

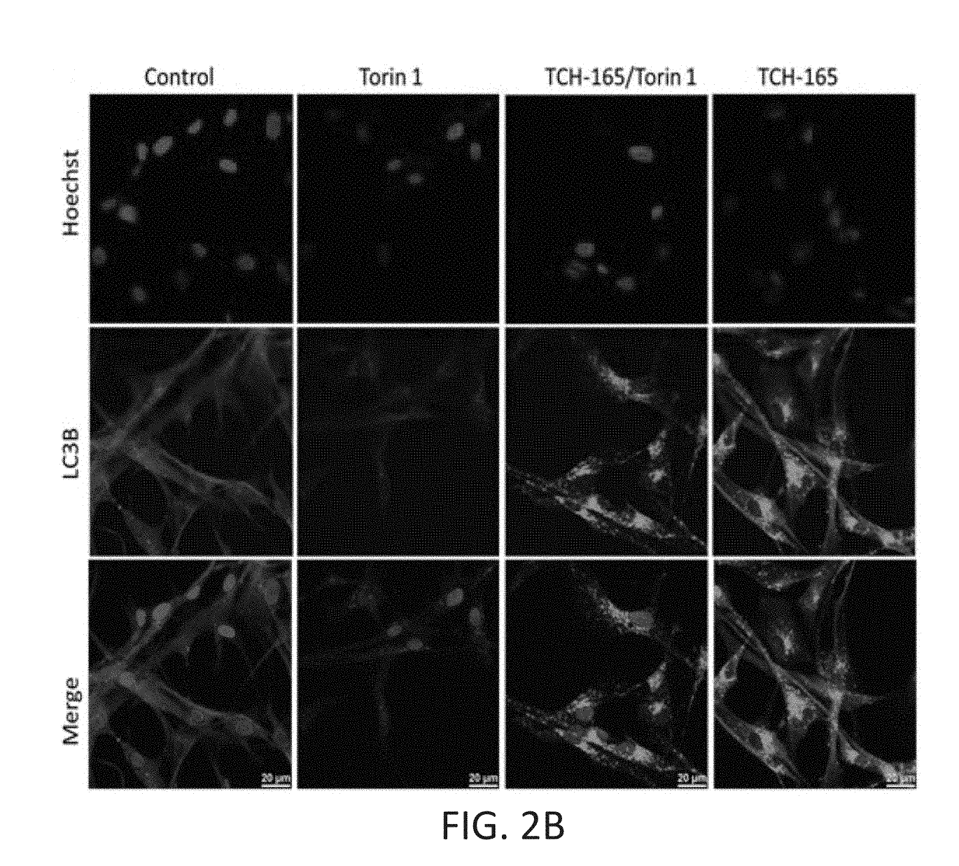

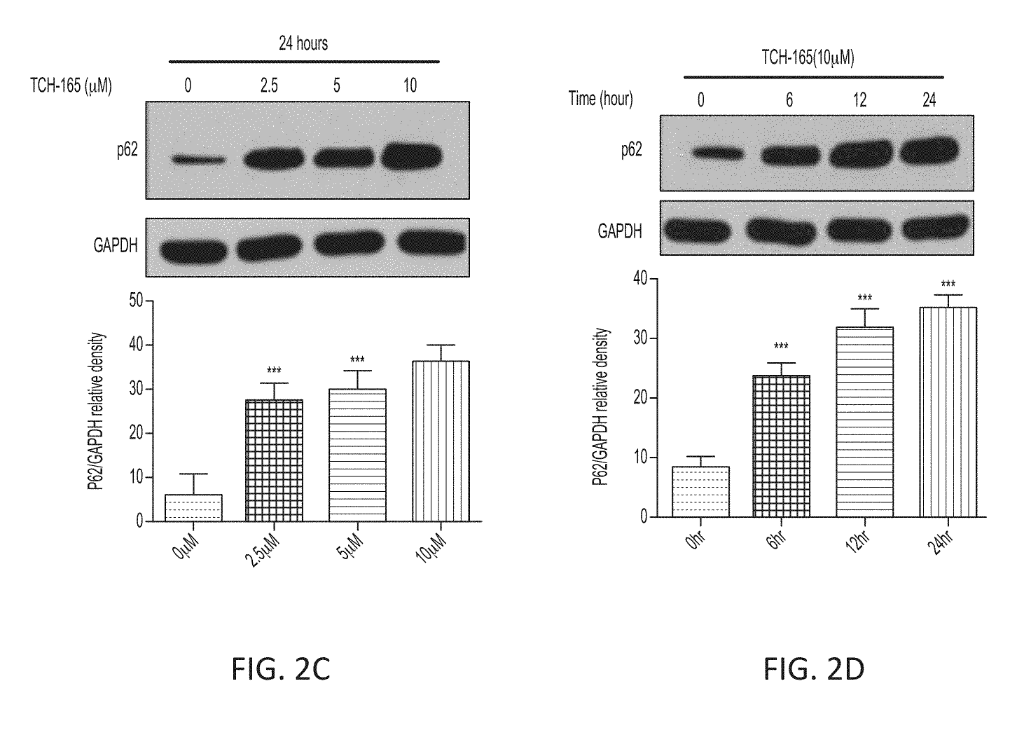

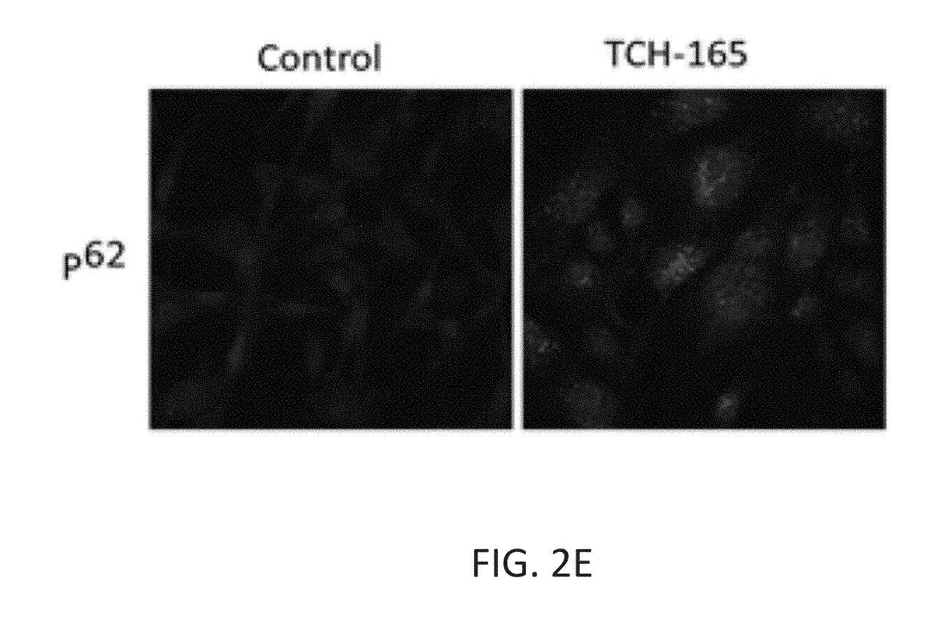

Treatment with BafA1 or TCH-165 led to a significant increase in the amount of LC3B-II (FIG. 2A). However, treatment with TCH-165 for 16 h followed by BafA1 for 4 hours did not further increase LC3B-II levels above that induced by TCH-165 alone (FIG. 2A), indicating that TCH-165 blocks autophagy flux.

To further evaluate its mechanism, TCH-165 was combined with the autophagy activator, torin1. Confocal immunofluorescence of LC3B in U87-MG cells was used to evaluate the effects of torin1 on TCH-165 inhibition of autophagy. U87-MG cells were treated with either vehicle or torin1 (100 nM) for 4 h, followed by TCH-165 (10 μM) for 16 h. LC3B polypeptides were stained with LC3B antibody/Alexa Fluor 488 secondary (green) and the cellular nuclei were stained with Hoechst DNA dye (blue).

As illustrated in FIG. 2B, treatment with torin1 alone induced modest accumulation of autophagic vacuoles, while the combination of TCH-165 with torin1 significantly increased the number of autophagic vacuoles (FIG. 2B). This suggests that TCH-165 blocks the degradation of autophagosome induced by torin1.

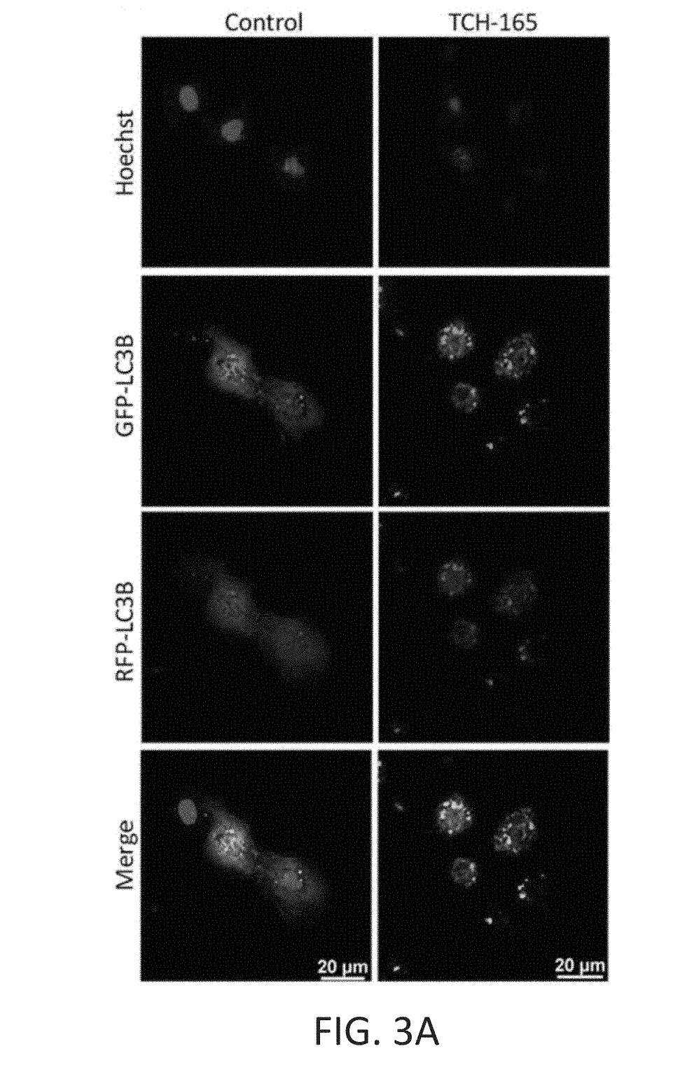

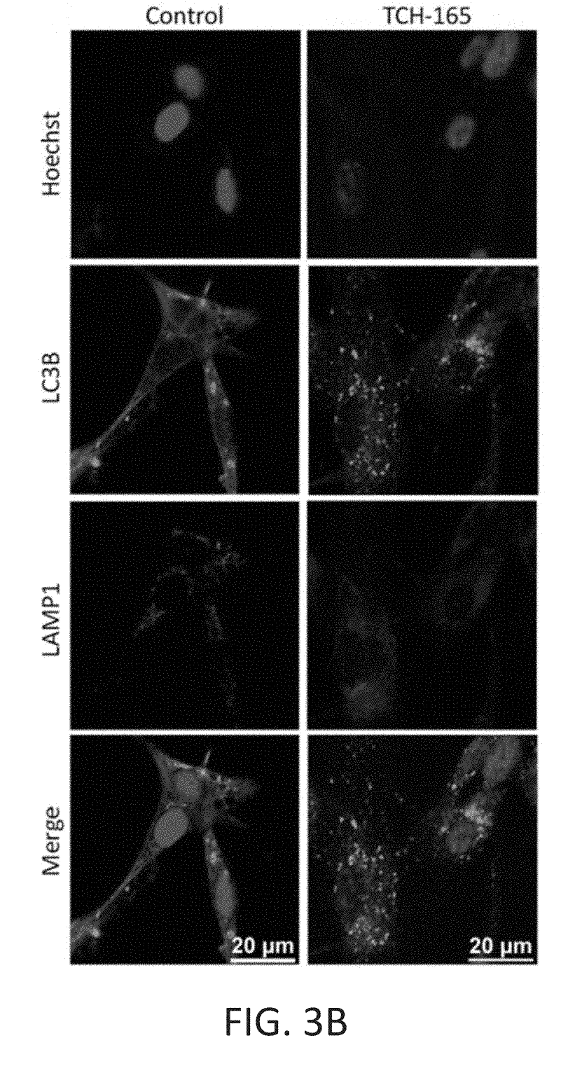

The effect of TCH-165 on the level of the autophagy substrate, p62, was then evaluated. During autophagy, p62/SQSTM1 is incorporated into autophagosome via the interaction with LC3B, where it serves as a receptor for the delivery of polyubiquitinated proteins to the autolysosome. The clearance of p62 therefore serves as a measure of autophagy flux (Bjorkoy et al., Methods Enzymol 452, 181-197 (2009)). U87-MG cells were treated with different concentrations of TCH-165 and immunoblotted with p62-specific antibody. GAPDH was immunoblotted as a loading control. Treatment of the cells with TCH-165 resulted in an increase in p62 in a TH-165 concentration (FIG. 2C) and time (FIG. 2D) dependent manner. These data were further confirmed by an increase in p62 punctae following TCH treatment, as detected by immunofluorescence imaging (FIG. 3E).

Increases in LC3B-II levels and accumulation of p62 show that TCH-165 is likely a late stage autophagy inhibitor, targeting either the lysosome and/or autophagosome-lysosome fusion.

### Example 3

### TCH-165 Inhibits Autophagasome-Lysosome Fusion

This Example describes experiments involving a tandem RFP-GFP-LC3B autophagy sensor to determine whether TCH-165 inhibits autophagy by interfering with autolysosome formation.

By combining an acid insensitive red fluorescent protein (RFP) with an acid sensitive green fluorescent protein (GFP), the conversion of autophagosomes (with neutral pH) to an autolysosome (with acidic pH) can be visualized by monitoring the specific loss of the GFP fluorescence upon acidification of the autophagosome following lysosomal fusion.

U87-MG cells were transduced with 30 particles per cell of tandem-RFP-GFP-LC3B and cultured for 24 h. Cells were then incubated with either vehicle or TCH-165 (10 μM) for an additional 24 h. Cells were fixed with 4% formaldehyde, counter stained with Hoechts DNA dye, and imaged on a Nikon C2 confocal microscope using standard filter sets for blue dye, GFP and RFP.

Cells expressing RFP-GFP-LC3B retained both GFP and RFP positive vacuoles upon TCH-165 treatment (FIG. 3A). These data indicate that TCH-165 inhibits autophagy flux by interfering with autolysosome formation.

However, GFP and RFP positive punctae could also represent de-acidified autolysosome, as seen with chloroquine treatment (data not shown). Co-localization of LC3B with LAMP1 was therefore examined to distinguish inhibition of autolysosome formation from autolysosome de-acidification. Co-localization of LC3B with LAMP1 occurs upon autolysosome formation.

U87MG cells were treated with either vehicle or TCH-165 (10 μM) for 24 h and immunostained with LC3B specific/Alexa Fluor 488 (green) antibody, LAMP1 specific/Alexa Fluor 594 antibody, Hoechst DNA dye (blue), or combinations thereof. Images were taken with Nikon A1 confocal microscope (60×).

Co-localization of LC3B and LAMP1 was not observed upon TCH-165 treatment, confirming that de-acidification was not involved (FIG. 3B).

These results were further confirmed by live imaging of lysosensor yellow/blue dextran in cells expressing LAMP1-RFP. Lysosensor yellow/blue dextran is a pH sensitive dye that is blue under acidic condition and yellow upon deprotonation (neutral pH). Lysosomes were distinguished from other acidic organelles by imaging with a combination of lysosensor yellow/blue dextran and LAM P1-RFP.

Unlike the lysosomotropic agent, chloroquine, which shifted the fluorescence to the yellow spectrum, TCH-165 treated cells mainly had red/blue vacuoles.

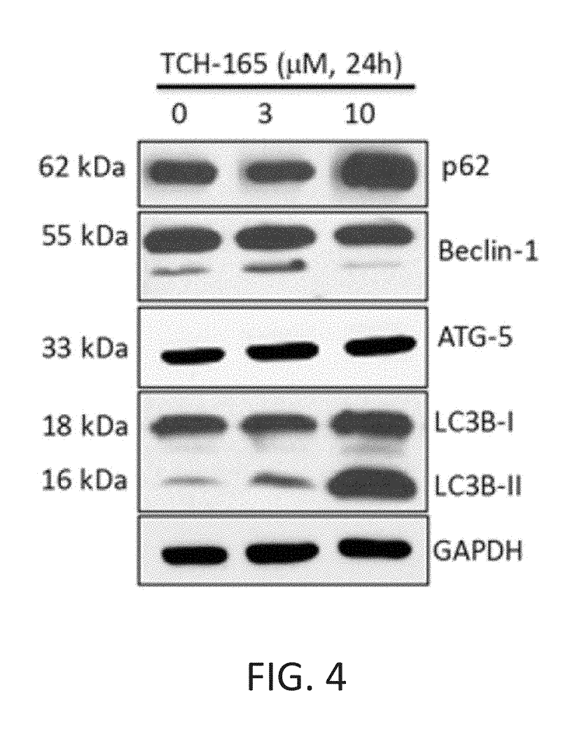

To evaluate whether TCH-165 as an effect of the expression of various genes, U87-MG cells were treated with TCH-165 for 24 h and cell lystaes were immunoblotted for LC3B, p62, beclin1 and ATGSFIG. TCH-165 did not appear to have any significant effect on the expression of other autophagic regulators like beclin-1, ATGS and ATG12 in U87-MG cells (FIG. 4).

### Example 4

### TCH-165 Inhibits Autophagy and Reduces Tumor Cell Viability

Glioblastoma cells exhibit high levels of basal autophagy. Some evidence suggests that the pro-survival function of autophagy supports tumor progression, metastatic dissemination and induces chemoresistance. The most widely used chemotherapeutic agent for glioblastoma multiform (GMB) is the DNA alkylating agent, temozolomide (TMZ), which can improve median patient survival to approximately 12-15 months, when treated with surgical resection and radiation.

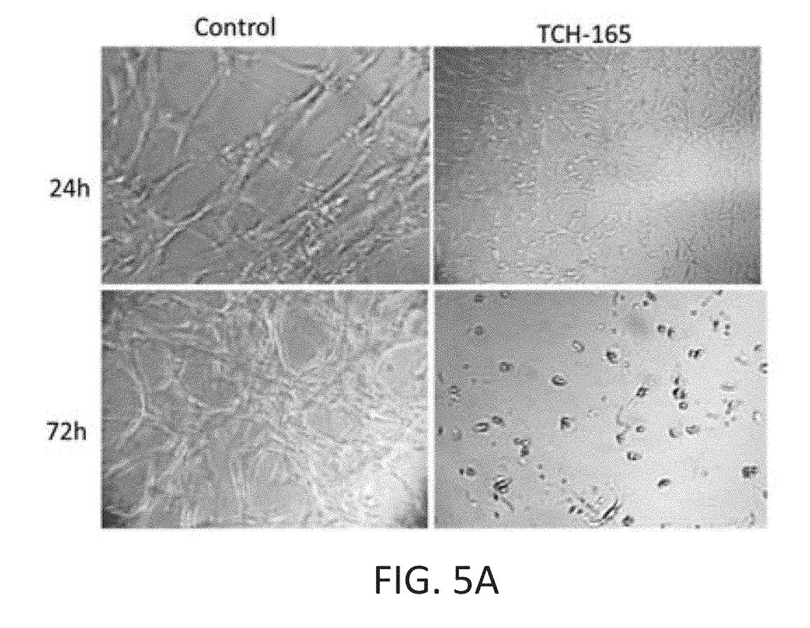

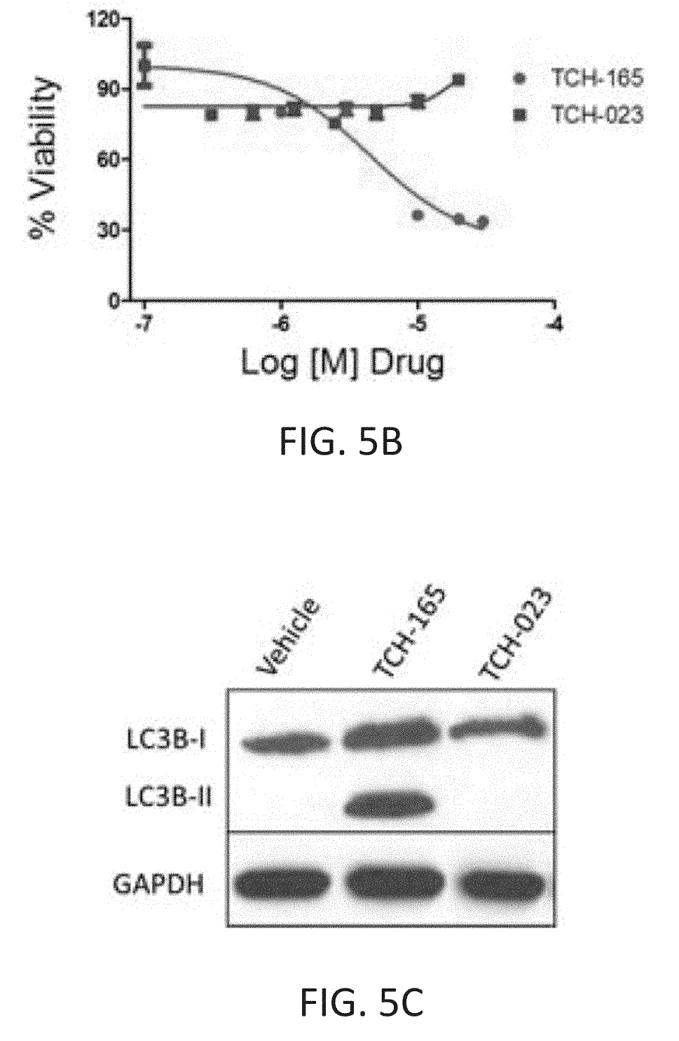

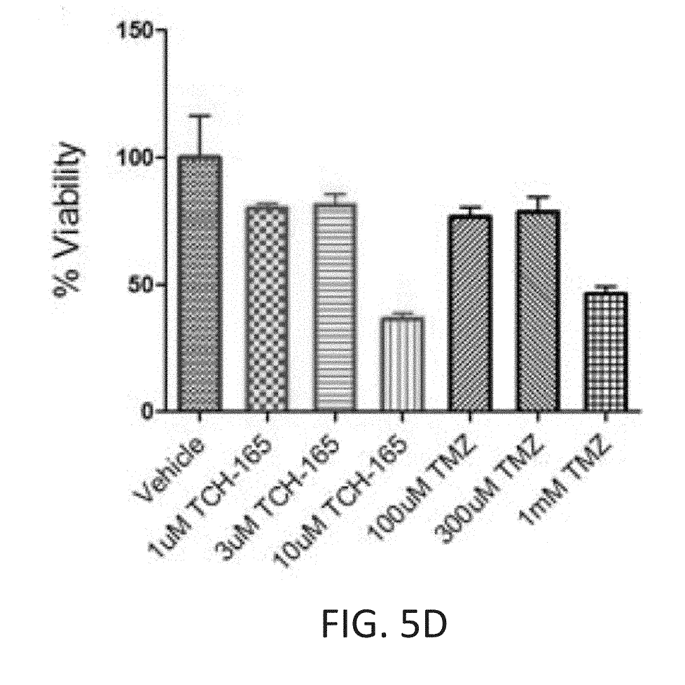

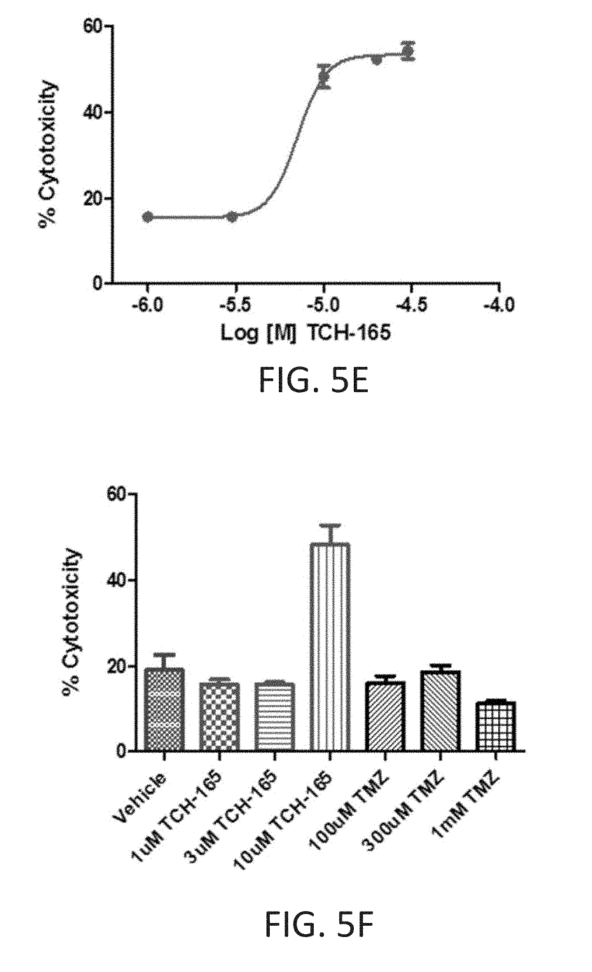

Treatment of glioblastoma cells such as U87-MG cells typically requires high concentrations (>0.1 mM) for marginal efficacy (˜20-30%) (Atif et al., PLoS One 10, e0131441 (2015)). As illustrated in FIG. 5D, treatment with up to 1 mM TMZ was required to kill 54% of U87-MG cells.

Considering the reliance on autophagy in glioblastoma cells, the effect of autophagy inhibition by TCH-165 on U87-MG cell viability was examined. Treatment of U87-MG with TCH-165, resulted a strong time (FIG. 5A) and concentration (FIG. 5B) dependent induction of cell death using either an MTS-based assay (FIG. 5B, GI50 4.5 μM) or an LDH-based assay (FIG. 5E-5F, GI50 7.0 μM) to measure cell viability. The concentrations required to induce cell death correlated well with concentrations required to inhibit autophagy. In addition, the structurally related TCH-023 did not inhibit autophagy (FIG. 5C) and subsequently did not induce any cell death, further supporting the conclusion that cell death is correlated with autophagy inhibition.

These data confirm that autophagy has a role in tumor survival. These data also indicate that inhibition of autophagasome-lysosome fusion may be an effective therapeutic strategy.

### Example 6

### pIC50 of Selected Imidazole Compounds

This Example illustrates that only low concentrations of imidazole compounds are needed to effectively inhibit growth of a variety of tumor types.

The compounds tested were TCH-165 and Imidazole 1 (also referred to as TCH-013).

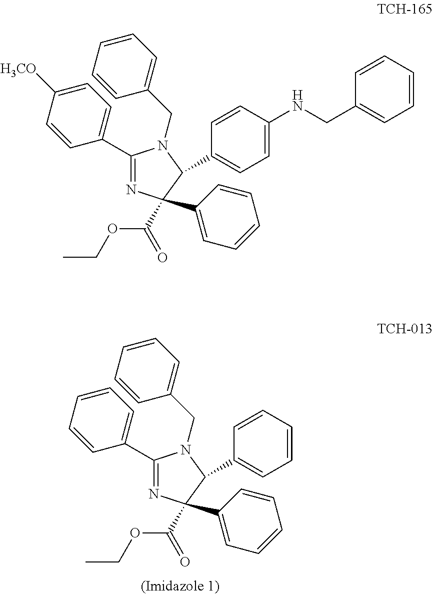 [SMILES: CCOC(=O)[C@]1(c2ccccc2)N=C(c2ccc(OC)cc2)N(Cc2ccccc2)[C@@H]1c1ccc(NCc2ccccc2)cc1 ; CCOC(=O)[C@]1(c2ccccc2)N=C(c2ccccc2)N(Cc2ccccc2)[C@@H]1c1ccccc1]

The pIC₅₀ values shown in Table 1, herein, was determined as the negative log of the amount of compound required to inhibit tumor cell growth by 50% (the IC₅₀). Thus, the larger the pIC₅₀ the more potent the compound. A pIC₅₀ of 4 indicates an IC₅₀ of 10-4. A commonly used cut-off for defining potent compounds is a pIC₅₀ greater than or equal to pIC₅₀ of 6, which is a compound with submicromolar potency.

TABLE 1

| | | | | pIC₅₀ |
|---|---|---|---|---|
| Species | Origin | Cell Line | TCH-013 | TCH-0165 |
| Canine | Histiocytic | HS BD | 5.3 | 5.2 |
| | Sarcoma | HS PJ | 5.4 | 5.5 |
| | | HS DH82 | 5.0 | 5.7 |
| | Osteosarcoma | cOS D17 | 4.7 | 5.2 |
| | | cOS Abrams | 4.8 | 4.9 |
| | | cOS Gracie | 5.2 | 5.7 |
| | | cOS Ginger | 5.5 | 5.1 |
| | | cOS Brandie | 5.0 | 5.5 |
| | | primary | | |
| | | cOS Brandie | 4.8 | 5.4 |
| | | mets | | |
| | | cOS Blaz | 4.9 | 5.2 |
| | Mastocytoma | MCT C2 | | 4.0 |
| | Normal | Normal FB1 | 3.7 | 3.7 |
| | Firbroblasts | Normal FB2 | 4.4 | 4.7 |
| | | Normal FB3 | 4.8 | |
| Feline | Squamous | Fel SCC1 | 5.0 | 5.7 |
| | Cell | Fel SCC2 | 3.7 | 4.6 |
| | Carcinoma | Fel SCC3 | 4.3 | 3.7 |
| Human | Osteosarcoma | hOS Saos2 | 5.3 | 5.5 |
| | | hOS U2OS | 4.8 | 5.3 |
| | | hOS MG63 | 4.7 | 5.5 |
| | Mammary | Hela cells | 5.0 | 5.1 |
| | Carcinoma | | | |
| | Embryonic | HEK293 | | 5.7 |
| | Kidney | | | |

The cell lines and species of origin and the pIC₅₀ of the compounds TCH-013 and TCH-0165 are presented in Table 1 herein. There is effective inhibition of growth with most tumor cell lines tested.

Three beagle dogs were tested for tolerance of the TCH165 compound, where the dosage was targeted to achieve about a 5× exposure than anticipated to be necessary for therapeutic efficacy. The three dogs tolerated the TCH165 compound very well without showing any clinical or biochemical signs of toxicity during a five-day tolerance study.

The following statements are intended to describe and summarize various embodiments according to the foregoing description in the specification.

### Statements:

(1) A method comprising administering a compound of formula I (or a salt thereof) to an animal:

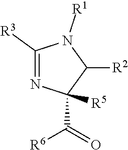

wherein:
R₁ is alkylaryl or arylalkylene, optionally substituted with 1-3 alkyl, alkoxy, halide, or CF₃ groups;
R₂ is phenyl or -phenyl-N(R₄)₂, wherein each R₄ is hydrogen, alkyl, aminoalkyl, formimideamide, thioalkyl, alkylenethiol, sulfonylamine, sulfonylaminoalkyl, aryl, arylalkylene, sulfonylalkylenearyl, cycloalkyl, alkylenecycloalkyl, or sulfonylcycloalkyl, wherein the aryl or cycloalkyl is optionally substituted with 1-3 hydroxy, alkoxy or halide groups;
R₃ is phenyl or benzyl optionally substituted with halide or alkoxy;
R₅ is phenyl; and
R₆ is alkoxy.

(2) The method of statement 1, wherein R₁ is phenylalkylene.

(3) The method of statement 1 or 2, wherein R₁ group is benzyl.

(4) The method of statement 1, 2, or 3, wherein R₂ is -phenyl-N(R₄)₂.

(5) The method of statement 1-3, or 4, wherein R₃ is phenyl optionally substituted with alkoxy.

(6) The method of statement 1-4, or 5, wherein R₆ is C1-C3 alkoxy.

(7) The method of statement 1-5, or 6, wherein compound of formula I is the TCH-165 compound:

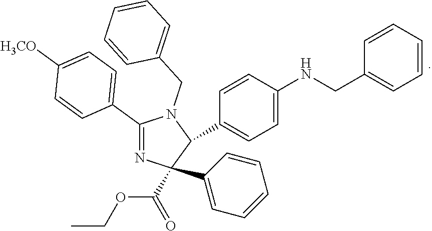 [SMILES: CCOC(=O)[C@]1(c2ccccc2)N=C(c2ccc(OC)cc2)N(Cc2ccccc2)[C@@H]1c1ccc(NCc2ccccc2)cc1]

(8) The method of statement 1, wherein the compound of formula I is Imidazole 1 (also referred to as the TCH-013 compound):

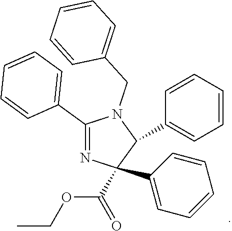 [SMILES: CCOC(=O)[C@]1(c2ccccc2)N=C(c2ccccc2)N(Cc2ccccc2)[C@@H]1c1ccccc1]

(9) The method of statement 1-7 or 8, wherein the compound is one or more of the following compounds:

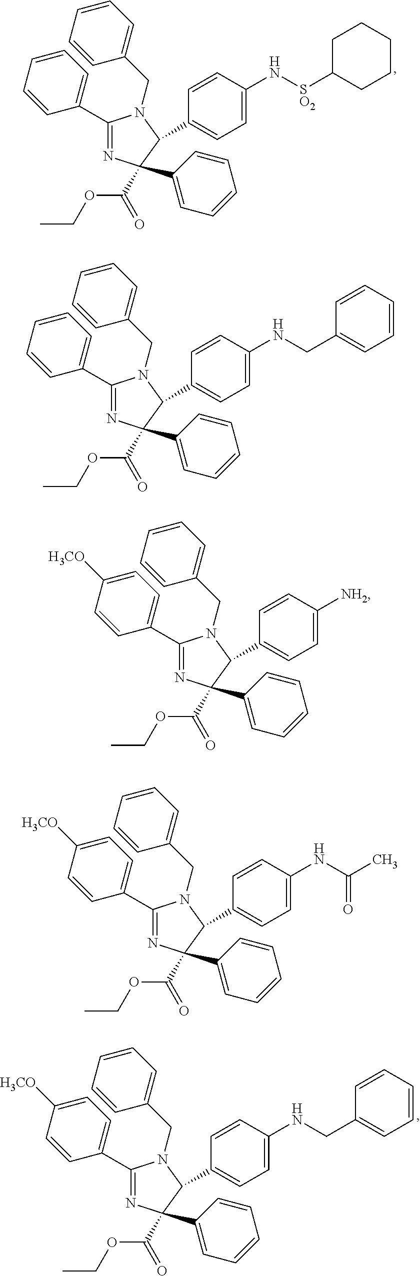

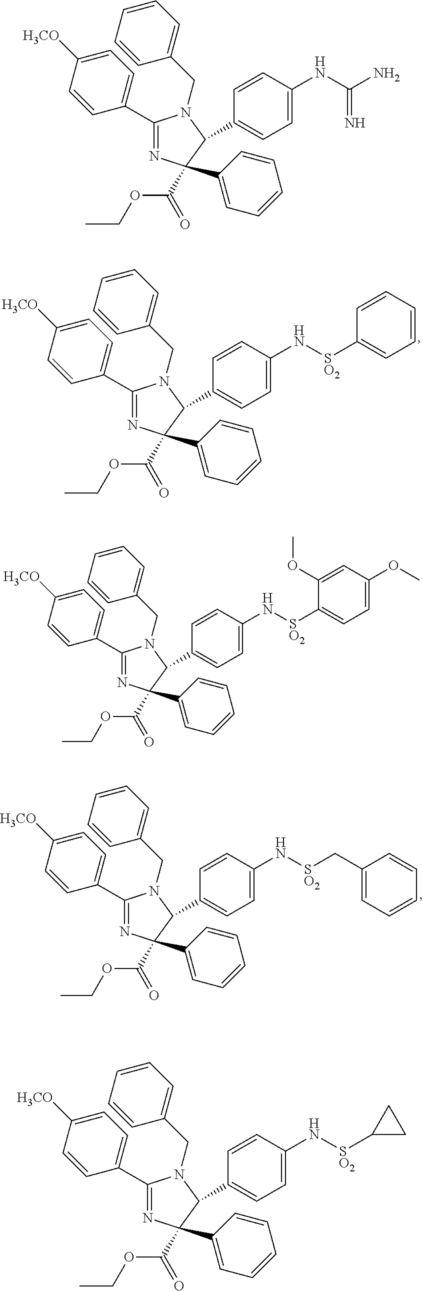 [SMILES: CCOC(=O)[C@]1(c2ccccc2)N=C(c2ccc(OC)cc2)N(Cc2ccccc2)[C@@H]1c1ccc(NC(=N)N)cc1 ; CCOC(=O)[C@]1(c2ccccc2)N=C(c2ccc(OC)cc2)N(Cc2ccccc2)[C@@H]1c1ccc(NS(=O)(=O)c2ccccc2)cc1 ; CCOC(=O)[C@]1(c2ccccc2)N=C(c2ccc(OC)cc2)N(Cc2ccccc2)[C@@H]1c1ccc(NS(=O)(=O)c2ccc(OC)cc2OC)cc1 ; CCOC(=O)[C@]1(c2ccccc2)N=C(c2ccc(OC)cc2)N(Cc2ccccc2)[C@@H]1c1ccc(NS(=O)(=O)Cc2ccccc2)cc1 ; CCOC(=O)[C@]1(c2ccccc2)N=C(c2ccc(OC)cc2)N(Cc2ccccc2)[C@@H]1c1ccc(NS(=O)(=O)C2CC2)cc1]

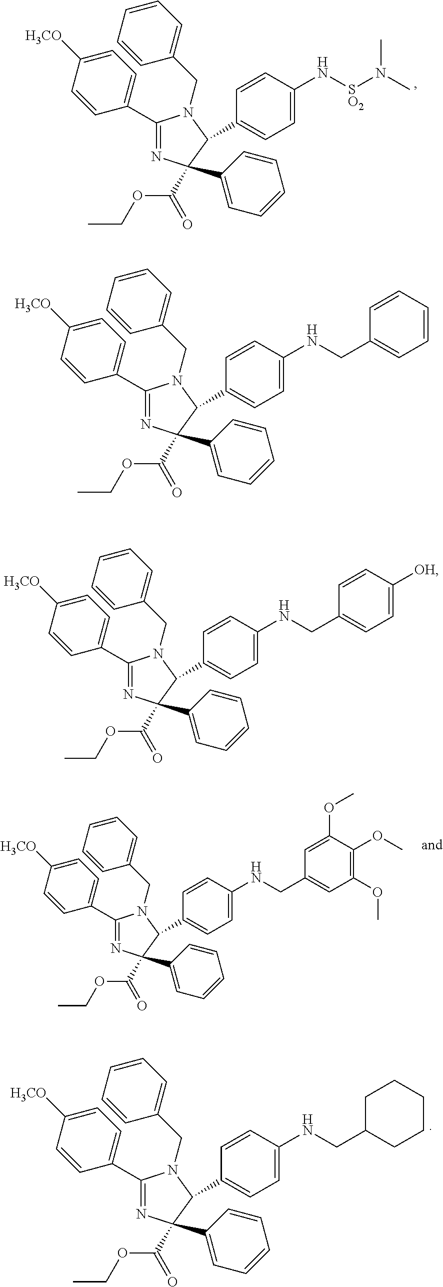 [SMILES: CCOC(=O)[C@]1(c2ccccc2)N=C(c2ccc(OC)cc2)N(Cc2ccccc2)[C@@H]1c1ccc(NS(=O)(=O)N(C)C)cc1 ; CCOC(=O)[C@]1(c2ccccc2)N=C(c2ccc(OC)cc2)N(Cc2ccccc2)[C@@H]1c1ccc(NCc2ccccc2)cc1 ; CCOC(=O)[C@]1(c2ccccc2)N=C(c2ccc(OC)cc2)N(Cc2ccccc2)[C@@H]1c1ccc(NCc2ccc(O)cc2)cc1 ; CCOC(=O)[C@]1(c2ccccc2)N=C(c2ccc(OC)cc2)N(Cc2ccccc2)[C@@H]1c1ccc(NCc2cc(OC)c(OC)c(OC)c2)cc1 ; "and" ; CCOC(=O)[C@]1(c2ccccc2)N=C(c2ccc(OC)cc2)N(Cc2ccccc2)[C@@H]1c1ccc(NCC2CCCCC2)cc1]

(10) The method of statement 1-8, or 9, wherein the animal is a human, a domesticated animal, or a zoo animal.

(11) The method of statement 1-9, or 10, wherein the animal is a dog, cat, bird, horse, alpaca, llama, camel, or elephant.

(12) The method of statement 1-10, or 11, wherein the animal is a dog.

(13) The method of statement 1-11, or 12, wherein the animal has cancer.

(14) The method of statement 1-12, or 13, wherein the animal has a malignant cancer.

(15) The method of statement 1-13, or 14, wherein the animal has a soft tissue cancer.

(16) The method of statement 1-14, or 15, wherein the animal has at least one solid tumor.

(17) The method of statement 1-15, or 16, wherein the animal has cancer selected from hematological cancer, lymphatic cancer, breast cancer, cervical cancer, ovarian cancer, prostate cancer, testicular cancer, pancreatic cancer, gastrointestinal cancer, neurological cancer, skin cancer, melanoma, bone cancer, or a combination thereof.

(18) The method of statement 1-16, or 17, wherein the animal has cancer selected from uterine cancer, urinary bladder cancer, soft tissue sarcoma, prostate cancer, primary peritoneal carcinoma, pancreatic cancer, ovarian cancer, esophageal cancer, malignant mesothelioma, lymphoma, lung cancer, kidney cancer, gastric cancer, gallbladder cancer, colorectal cancer, breast cancer, glioblastoma, glioblastoma astrocytoma, histiocytic sarcoma, lymphoma, osteosarcoma, or a combination thereof.

(19) The method of statement 1-17, or 18, wherein the animal has cancer selected from glioblastoma, glioblastoma astrocytoma, osteosarcoma, histiocytic sarcoma, mastocytoma, lymphoma, sarcoma, or a combination thereof.

(20) A composition comprising a carrier and a compound of formula I:

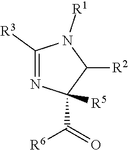

wherein:
R₁ is alkylaryl or arylalkylene, optionally substituted with 1-3 alkyl, alkoxy, halide, or CF₃ groups;
R₂ is phenyl or -phenyl-N(R₄)₂, wherein each R₄ is hydrogen, alkyl, aminoalkyl, formimideamide, thioalkyl, alkylenethiol, sulfonylamine, sulfonylaminoalkyl, aryl, arylalkylene, sulfonylalkylenearyl, cycloalkyl, alkylenecycloalkyl, or sulfonylcycloalkyl, wherein the aryl or cycloalkyl is optionally substituted with 1-3 hydroxy, alkoxy or halide groups;
R₃ is phenyl or benzyl optionally substituted with halide or alkoxy;
R₅ is phenyl; and
R₆ is alkoxy.

(21) The composition of statement 20, wherein R₁ is phenylalkylene.

(22) The composition of statement 20 or 21, wherein R₁ group is benzyl.

(23) The composition of statement 20, 21, or 22, wherein R₂ is —phenyl-N(R₄)₂.

(24) The composition of statement 20-22, or 23, wherein R₃ is phenyl optionally substituted with alkoxy.

(25) The composition of statement 20-23, or 24, wherein R₆ is C1-C3 alkoxy.

(26) The composition of statement 20-24, or 25, wherein compound of formula I is the TCH-165 compound:

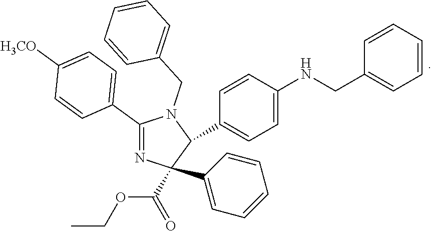 [SMILES: CCOC(=O)[C@]1(c2ccccc2)N=C(c2ccc(OC)cc2)N(Cc2ccccc2)[C@@H]1c1ccc(NCc2ccccc2)cc1]

(27) The composition of statement 20-25, or 26, wherein the compound of formula I is Imidazole 1 (also referred to as the TCH-013 compound):

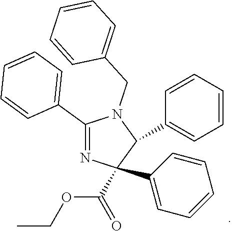 [SMILES: CCOC(=O)[C@]1(c2ccccc2)N=C(c2ccccc2)N(Cc2ccccc2)[C@@H]1c1ccccc1]

(28) The composition of statement 20-26 or 27, wherein the compound is one or more of the following compounds:

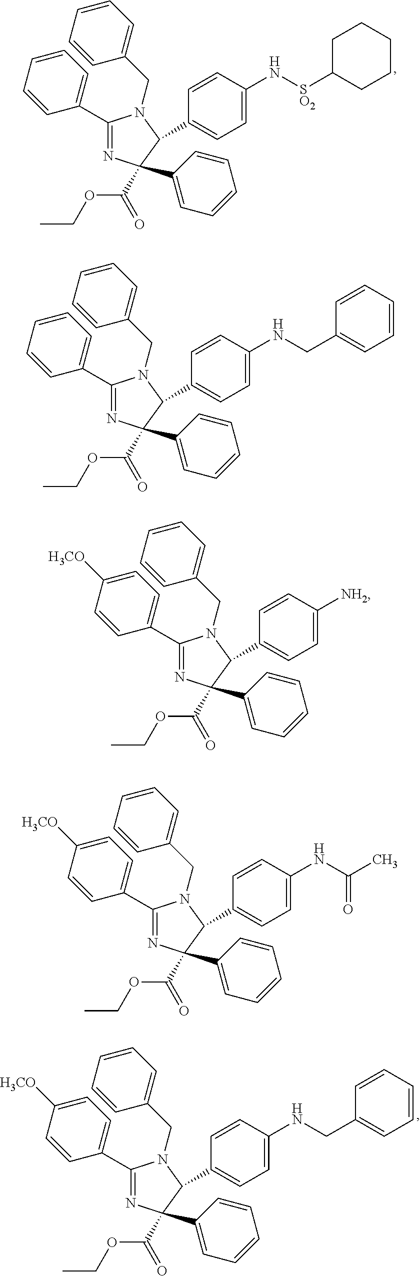

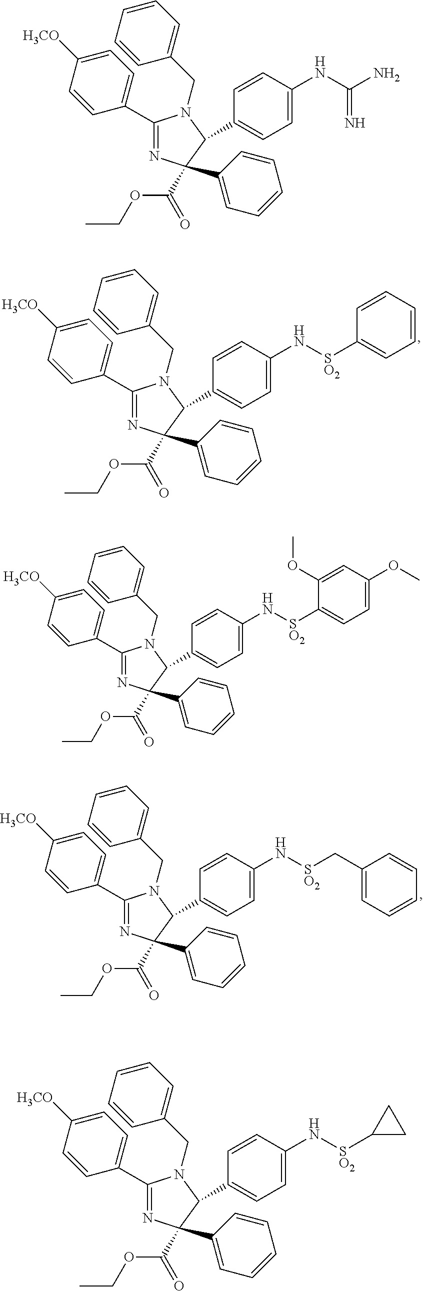 [SMILES: CCOC(=O)[C@]1(c2ccccc2)N=C(c2ccc(OC)cc2)N(Cc2ccccc2)[C@@H]1c1ccc(NC(=N)N)cc1 ; CCOC(=O)[C@]1(c2ccccc2)N=C(c2ccc(OC)cc2)N(Cc2ccccc2)[C@@H]1c1ccc(NS(=O)(=O)c2ccccc2)cc1 ; CCOC(=O)[C@]1(c2ccccc2)N=C(c2ccc(OC)cc2)N(Cc2ccccc2)[C@@H]1c1ccc(NS(=O)(=O)c2ccc(OC)cc2OC)cc1 ; CCOC(=O)[C@]1(c2ccccc2)N=C(c2ccc(OC)cc2)N(Cc2ccccc2)[C@@H]1c1ccc(NS(=O)(=O)Cc2ccccc2)cc1 ; CCOC(=O)[C@]1(c2ccccc2)N=C(c2ccc(OC)cc2)N(Cc2ccccc2)[C@@H]1c1ccc(NS(=O)(=O)C2CC2)cc1]

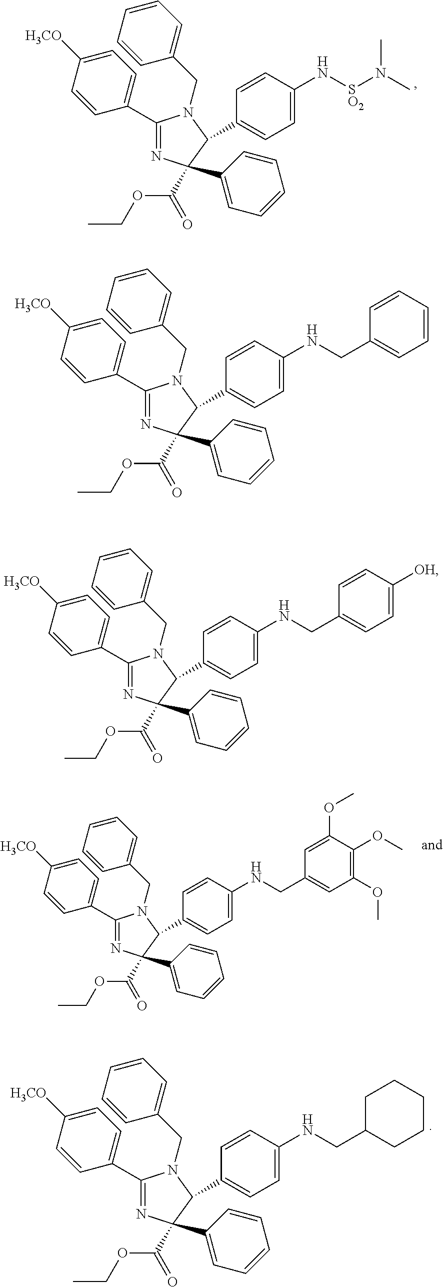 [SMILES: CCOC(=O)[C@]1(c2ccccc2)N=C(c2ccc(OC)cc2)N(Cc2ccccc2)[C@@H]1c1ccc(NS(=O)(=O)N(C)C)cc1 ; CCOC(=O)[C@]1(c2ccccc2)N=C(c2ccc(OC)cc2)N(Cc2ccccc2)[C@@H]1c1ccc(NCc2ccccc2)cc1 ; CCOC(=O)[C@]1(c2ccccc2)N=C(c2ccc(OC)cc2)N(Cc2ccccc2)[C@@H]1c1ccc(NCc2ccc(O)cc2)cc1 ; CCOC(=O)[C@]1(c2ccccc2)N=C(c2ccc(OC)cc2)N(Cc2ccccc2)[C@@H]1c1ccc(NCc2cc(OC)c(OC)c(OC)c2)cc1 ; "and" ; CCOC(=O)[C@]1(c2ccccc2)N=C(c2ccc(OC)cc2)N(Cc2ccccc2)[C@@H]1c1ccc(NCC2CCCCC2)cc1]

The specific compositions and methods described herein are representative, exemplary and not intended as limitations on the scope of the instant disclosure. Other objects, aspects, and embodiments will occur to those skilled in the art upon consideration of this specification and are encompassed within the spirit of the instant disclosure as defined by the scope of the claims. It will be readily apparent to one skilled in the art that varying substitutions and modifications may be made without departing from the scope and spirit of the instant disclosure. The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the instant disclosure has been specifically disclosed by embodiments and optional features, modification and variation of the concepts herein disclosed may be resorted to by those skilled in the art, and that such modifications and variations are considered to be within the scope of this disclosure as defined by the appended claims and statements.

The invention illustratively described herein may be practiced in the absence of any element or elements, or limitation or limitations, which is not specifically disclosed herein as essential. The methods and processes illustratively described herein may be practiced in differing orders of steps, and the methods and processes are not necessarily restricted to the orders of steps indicated herein or in the claims.

As used herein and in the appended claims, the singular forms “a,” “an,” and “the” include plural reference unless the context clearly dictates otherwise. Thus, for example, a reference to “a cell” or “a compound” or “an animal” includes a plurality of such cells, compounds, or animals, and so forth. In this document, the term “or” is used to refer to a nonexclusive or, such that “A or B” includes “A but not B,” “B but not A,” and “A and B,” unless otherwise indicated.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group.

The Abstract is provided to comply with 37 C.F.R. § 1.72(b) to allow the reader to quickly ascertain the nature and gist of the technical disclosure. The Abstract is submitted with the understanding that it will not be used to interpret or limit the scope or meaning of the claims.

What is claimed is:

1. A method for treating cancer comprising administering a compound of formula I, or a salt thereof, to an animal in need thereof: 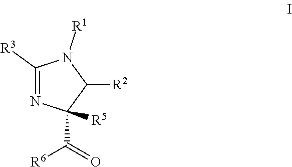
wherein:
R¹ is alkylaryl or arylalkylene, optionally substituted with one to three (1-3) alkyl, alkoxy, halide, or CF₃ groups;
R² is phenyl or -phenyl-N(R⁴)₂, wherein each R⁴ is independently hydrogen, acyl, alkyl, aminoalkyl, formimideamide, thioalkyl, alkylenethiol, aminosulfonyl, sulfonylamine, sulfonylaminoalkyl, aryl, arylalkylene, arylsulfonyl, sulfonylalkylenearyl, cycloalkyl, alkylenecycloalkyl, cycloalkylalkyl, cycloalkylsulfonyl or sulfonylcycloalkyl, wherein the aryl or cycloalkyl is optionally substituted with one to three (1-3) hydroxy, alkoxy or halide groups or
R⁴, and the nitrogen to which it is attached, form a guinidinyl group;
R³ is phenyl or benzyl optionally substituted with halide or alkoxy;
R⁵ is phenyl; and
R⁶ is alkoxy.

2. The method of claim 1, wherein R₁ is phenylalkyl.

3. The method of claim 1, wherein R₁ group is benzyl.

4. The method of claim 1, wherein R₂ is -phenyl-N(R₄)₂.

5. The method of claim 1, wherein R² is -phenyl-N(H)benzyl; -phenyl-N(H)C(O)alkyl; -phenyl-N(H)C(NH)NH₂; -phenyl-N(H)SO₂phenyl; -phenyl-N(H)SO₂dialkoxyphenyl; -phenyl-N(H)SO₂benzyl; -phenyl-N(H)SO₂cycloalkyl; or -phenyl-N(H)SO₂N(alkyl)₂.

6. The method of claim 1, wherein R₃ is phenyl optionally substituted with alkoxy.

7. The method of claim 1, wherein R₆ is C₁-C₃ alkoxy.

8. The method of claim 1, wherein compound of formula I is a compound having the formula:

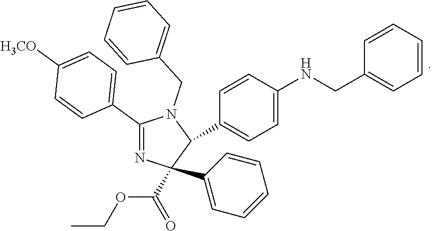 [SMILES: CCOC(=O)[C@]1(c2ccccc2)N=C(c2ccc(OC)cc2)N(Cc2ccccc2)[C@@H]1c1ccc(NCc2ccccc2)cc1]

9. The method of claim 1, wherein the compound of formula I is a compound having the formula:

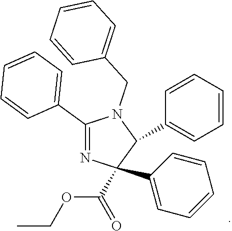 [SMILES: CCOC(=O)[C@]1(c2ccccc2)N=C(c2ccccc2)N(Cc2ccccc2)[C@@H]1c1ccccc1]

10. The method of claim 1, wherein the compound is one or more of the following compounds:

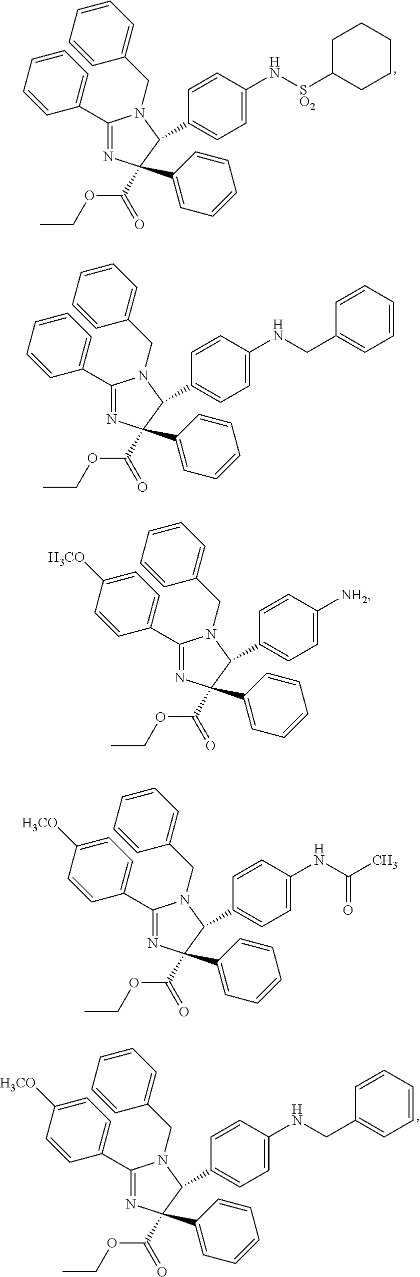

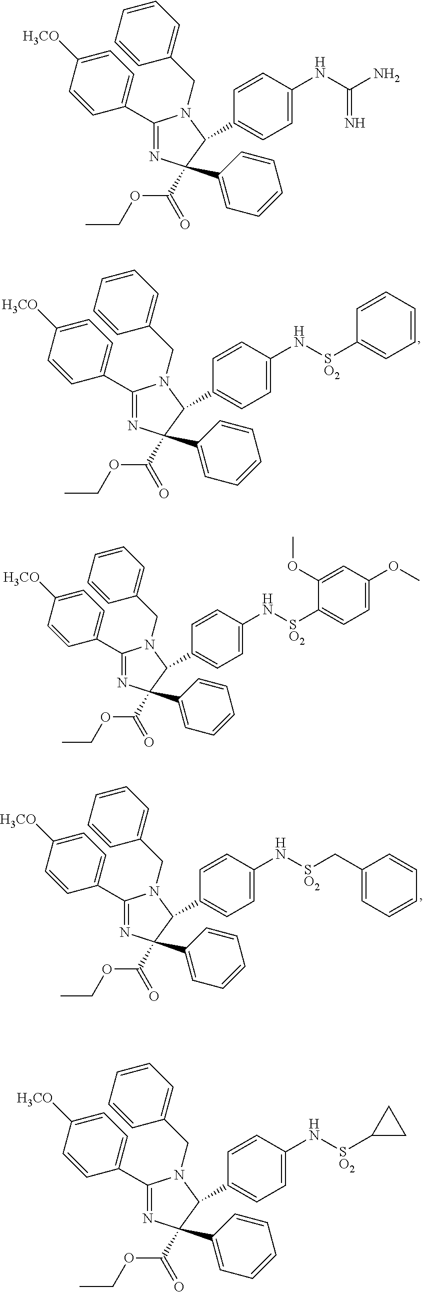 [SMILES: CCOC(=O)[C@]1(c2ccccc2)N=C(c2ccc(OC)cc2)N(Cc2ccccc2)[C@@H]1c1ccc(NC(=N)N)cc1 ; CCOC(=O)[C@]1(c2ccccc2)N=C(c2ccc(OC)cc2)N(Cc2ccccc2)[C@@H]1c1ccc(NS(=O)(=O)c2ccccc2)cc1 ; CCOC(=O)[C@]1(c2ccccc2)N=C(c2ccc(OC)cc2)N(Cc2ccccc2)[C@@H]1c1ccc(NS(=O)(=O)c2ccc(OC)cc2OC)cc1 ; CCOC(=O)[C@]1(c2ccccc2)N=C(c2ccc(OC)cc2)N(Cc2ccccc2)[C@@H]1c1ccc(NS(=O)(=O)Cc2ccccc2)cc1 ; CCOC(=O)[C@]1(c2ccccc2)N=C(c2ccc(OC)cc2)N(Cc2ccccc2)[C@@H]1c1ccc(NS(=O)(=O)C2CC2)cc1]

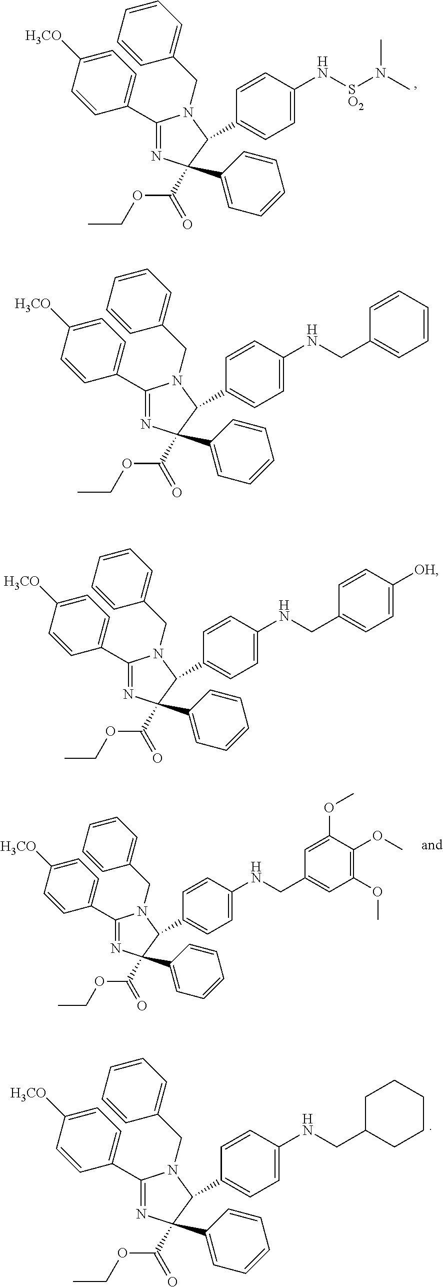 [SMILES: CCOC(=O)[C@]1(c2ccccc2)N=C(c2ccc(OC)cc2)N(Cc2ccccc2)[C@@H]1c1ccc(NS(=O)(=O)N(C)C)cc1 ; CCOC(=O)[C@]1(c2ccccc2)N=C(c2ccc(OC)cc2)N(Cc2ccccc2)[C@@H]1c1ccc(NCc2ccccc2)cc1 ; CCOC(=O)[C@]1(c2ccccc2)N=C(c2ccc(OC)cc2)N(Cc2ccccc2)[C@@H]1c1ccc(NCc2ccc(O)cc2)cc1 ; CCOC(=O)[C@]1(c2ccccc2)N=C(c2ccc(OC)cc2)N(Cc2ccccc2)[C@@H]1c1ccc(NCc2cc(OC)c(OC)c(OC)c2)cc1 ; "and" ; CCOC(=O)[C@]1(c2ccccc2)N=C(c2ccc(OC)cc2)N(Cc2ccccc2)[C@@H]1c1ccc(NCC2CCCCC2)cc1]

11. The method of claim 1, wherein the animal is a human, a domesticated animal, or a zoo animal.

12. The method of claim 1, wherein the animal is a dog, cat, bird, horse, alpaca, llama, camel, or elephant.

13. The method of claim 1, wherein the animal is a dog.

14. The method of claim 1, wherein the cancer is a malignant cancer.

15. The method of claim 1, wherein the cancer is a soft tissue cancer.

16. The method of claim 1, wherein the cancer is at least one solid tumor.

17. The method of claim 1, wherein the cancer is hematological cancer, lymphatic cancer, breast cancer, cervical cancer, ovarian cancer, prostate cancer, testicular cancer, pancreatic cancer, gastrointestinal cancer, neurological cancer, skin cancer, melanoma, bone cancer, or a combination thereof.

18. The method of claim 1, wherein the cancer is uterine cancer, urinary bladder cancer, soft tissue sarcoma, prostate cancer, primary peritoneal carcinoma, pancreatic cancer, ovarian cancer, esophageal cancer, malignant mesothelioma, lymphoma, lung cancer, kidney cancer, gastric cancer, gallbladder cancer, colorectal cancer, breast cancer, glioblastoma, glioblastoma astrocytoma, histiocytic sarcoma, lymphoma, osteosarcoma, or a combination thereof.

19. The method of claim 1, wherein the cancer is glioblastoma, glioblastoma astrocytoma, osteosarcoma, histiocytic sarcoma, mastocytoma, lymphoma, sarcoma, or a combination thereof.
